SEQUENCE LISTING (56 sequences)

SEQ ID NO: 1 - DNA, 20 bp, Artificial Sequence
  sgRNA sequence
tgtcaggtaataaaaataag

SEQ ID NO: 2 - DNA, 20 bp, Artificial Sequence
  sgRNA sequence
tattggttaaaaaagataca

SEQ ID NO: 3 - DNA, 20 bp, Artificial Sequence
  sgRNA sequence
agtcagtgaaactatcctcg

SEQ ID NO: 4 - DNA, 20 bp, Artificial Sequence
  sgRNA sequence
gtcaactgagattatccagt

SEQ ID NO: 5 - DNA, 20 bp, Artificial Sequence
  sgRNA sequence
tgttcctcagaaatgaagga

SEQ ID NO: 6 - DNA, 20 bp, Artificial Sequence
  sgRNA sequence
ctatgtactttcctttacca

SEQ ID NO: 7 - DNA, 20 bp, Artificial Sequence
  sgRNA sequence
acagcaaaagaaactaccat

SEQ ID NO: 8 - DNA, 20 bp, Artificial Sequence
  sgRNA sequence
gcatgttctcactcataggt

SEQ ID NO: 9 - DNA, 20 bp, Artificial Sequence
  sgRNA sequence
gctgttgtacactagatctc

SEQ ID NO: 10 - DNA, 20 bp, Artificial Sequence
  sgRNA sequence
aatagtaatataaattaggg

SEQ ID NO: 11 - DNA, 20 bp, Artificial Sequence
  sgRNA sequence
agagatatagatcaatggaa

SEQ ID NO: 12 - DNA, 20 bp, Artificial Sequence
  sgRNA sequence
ggctagccatatgtaaaaag

SEQ ID NO: 13 - DNA, 20 bp, Artificial Sequence
  sgRNA sequence
atagcactaaatgcttacat

SEQ ID NO: 14 - DNA, 20 bp, Artificial Sequence
  sgRNA sequence
gctatctgtacaaaatggaa

SEQ ID NO: 15 - DNA, 20 bp, Artificial Sequence
  sgRNA sequence
gttcaagcagatttgaacag

SEQ ID NO: 16 - DNA, 20 bp, Artificial Sequence
  sgRNA sequence
tattatacaaagtagccaaa

SEQ ID NO: 17 - DNA, 20 bp, Artificial Sequence
  sgRNA sequence
agattcaatgaaataaaaaa

SEQ ID NO: 18 - DNA, 20 bp, Artificial Sequence
  sgRNA sequence
gtagtgaaaggaacaccaga

SEQ ID NO: 19 - DNA, 20 bp, Artificial Sequence
  sgRNA sequence
tgtggcataaagactctctg

SEQ ID NO: 20 - DNA, 20 bp, Artificial Sequence
  sgRNA sequence
tgtatactcagaaaagacct

SEQ ID NO: 21 - DNA, 20 bp, Artificial Sequence
  sgRNA sequence
acaccagtaacagactaaca

SEQ ID NO: 22 - DNA, 20 bp, Artificial Sequence
  sgRNA sequence
atagtgttggaagttctggc

SEQ ID NO: 23 - DNA, 20 bp, Artificial Sequence
  sgRNA sequence
ttaaaacagcaatacctgag

SEQ ID NO: 24 - DNA, 20 bp, Artificial Sequence
  sgRNA sequence
tctctggagtatatacctag

SEQ ID NO: 25 - DNA, 20 bp, Artificial Sequence
  sgRNA sequence
gtatcgctataaatttccat

SEQ ID NO: 26 - DNA, 20 bp, Artificial Sequence
  sgRNA sequence
aaacaactaatagttcacag

SEQ ID NO: 27 - DNA, 20 bp, Artificial Sequence
  sgRNA sequence
ggatgtgcttagtttcacag

SEQ ID NO: 28 - DNA, 20 bp, Artificial Sequence
  sgRNA sequence
ttactctgcaatacaccaag

SEQ ID NO: 29 - DNA, 20 bp, Artificial Sequence
  sgRNA sequence
gctaggagatacagttctga

SEQ ID NO: 30 - DNA, 20 bp, Artificial Sequence
  sgRNA sequence
gctatttcattaagtcaccc

SEQ ID NO: 31 - DNA, 20 bp, Artificial Sequence
  sgRNA sequence
gagccccttgaataaaggaa

SEQ ID NO: 32 - DNA, 20 bp, Artificial Sequence
  sgRNA sequence
gcatggtgcgggaagacgac

SEQ ID NO: 33 - DNA, 22 bp, Artificial Sequence
  miR target sequence
agtgaattctaccagtgccata

SEQ ID NO: 34 - DNA, 24 bp, Artificial Sequence
  miR target sequence
agtgtgagttctaccattgccaaa

SEQ ID NO: 35 - DNA, 20 bp, Mus musculus
ttgcccaacctctcaaacgt

SEQ ID NO: 36 - DNA, 20 bp, Artificial Sequence
  primer sequence
cctagaattcagggccgagg

SEQ ID NO: 37 - DNA, 20 bp, Artificial Sequence
  primer sequence
ttggttgaaacagggaaggc

SEQ ID NO: 38 - DNA, 27 bp, Artificial Sequence
  primer sequence
agctgcccaagcacttatagacatgac

SEQ ID NO: 39 - DNA, 50 bp, Artificial Sequence
  primer sequence
cctctctatgggcagtcggtgaaaaatctccctagaattcagggccgagg

SEQ ID NO: 40 - DNA, 28 bp, Artificial Sequence
  primer sequence
atttttacgtttgttccctccatcttcc

SEQ ID NO: 41 - DNA, 52 bp, Artificial Sequence
  primer sequence
cctctctatgggcagtcggtgaacctacacatttggttgaaacagggaaggc

SEQ ID NO: 42 - DNA, 20 bp, Artificial Sequence
  target sequence
catgaaggtaaccactactg

SEQ ID NO: 43 - DNA, 20 bp, Artificial Sequence
  target sequence
cctggccttccctgtatcaa

SEQ ID NO: 44 - DNA, 20 bp, Artificial Sequence
  target sequence
caaatttgggccttggtgtc

SEQ ID NO: 45 - DNA, 19 bp, Artificial Sequence
  target sequence
gaatgttgggatctcaaac

SEQ ID NO: 46 - DNA, 21 bp, Artificial Sequence
  target sequence
gtgctgttttgcaaaccttta

SEQ ID NO: 47 - DNA, 20 bp, Artificial Sequence
  target sequence
tccttgggcctcctatgtca

SEQ ID NO: 48 - DNA, 20 bp, Artificial Sequence
  target sequence
ttgcccaacctctcaaacgt

SEQ ID NO: 49 - DNA, 20 bp, Artificial Sequence
  target sequence
caggccttggtgttatgaat

SEQ ID NO: 50 - DNA, 20 bp, Artificial Sequence
  target sequence
ttggtacttacaaggccatg

SEQ ID NO: 51 - DNA, 19 bp, Artificial Sequence
  target sequence
tcacacaagagccacccca

SEQ ID NO: 52 - DNA, 20 bp, Artificial Sequence
  target sequence
ctgaggcccatccatggtca

SEQ ID NO: 53 - DNA, 21 bp, Artificial Sequence
  target sequence
gcctcatgtgaccctgtgacc

SEQ ID NO: 54 - DNA, 2211 bp, adeno-associated virus hu68
  CDS (1)..(2211): AAVhu68 Capsid
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 55 - protein, 736 aa, adeno-associated virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 56 - DNA, 20 bp, Artificial Sequence
  non-targeted sequence
gagacggtcttcgacgtctc

(19) United States
(12) Patent Application Publication
(10) Pub. No.: US 2023/0167438 A1
(43) Pub. Date: Jun. 1, 2023

(54) COMPOSITIONS AND USES THEREOF FOR TREATMENT OF ANGELMAN SYNDROME

(71) Applicant: The Trustees of the University of Pennsylvania, Philadelphia, PA (US)

(72) Inventors: James M. Wilson, Philadelphia, PA (US); Ralf Schmid, Havertown, PA (US)

(21) Appl. No.: 17/997,004

(22) PCT Filed: Apr. 27, 2021
(86) PCT No.: PCT/US2021/029378

§ 371 Date: Oct. 24, 2022

Related U.S. Application Data
(60) Provisional application No. 63/118,299, filed on Nov. 25, 2020.
(60) Provisional application No. 63/016,712, filed on Apr. 28, 2020.

(51) Int. Cl. C12N 15/11; C12N 15/86; C12N 9/22; C12N 15/90; A61P 25/00

(52) U.S. Cl. CPC C12N 15/11; C12N 15/86; C12N 9/22; C12N 15/907; A61P 25/00; C12N 2310/20; C12N 2800/80; C12N 2750/14143; C12N 2750/14145; A61K 48/00

(57) ABSTRACT

A composition comprising an expression cassette having a nucleic acid sequence encoding one or more elements of a gene editing system that targets UBE3A-ATS on a paternal allele in a neuron of a patient having Angelman syndrome is provided. Also provided is a method for treating one or more symptoms of Angelman syndrome (AS) in a patient having deficient UBE3A expression in neurons, wherein the method comprises delivering a nucleic acid sequence that encodes one or more elements of a gene editing system that targets UBE3A-ATS to modify the UBE3A-ATS coding sequence and provide for expression of paternal UBE3A.

### BACKGROUND OF THE INVENTION

Angelman syndrome (AS) is a rare, severe neurodevelopmental disorder. Characteristic symptoms include delayed development, intellectual disability, severe speech impairment, problems with movement and balance (ataxia) and often early-onset recurrent seizures (epilepsy). There is currently no curative therapy available for AS.

Development of AS results from the lack of UBE3A (Ubiquitin-protein ligase E3A, also known as E6AP ubiquitin-protein ligase) protein expression in neurons. UBE3A is only expressed monoallelically from the maternally inherited allele in neurons, whereas the paternally inherited UBE3A allele is silenced in neurons. Individuals affected by AS have large deletions or loss-of-function mutations within the UBE3A gene located on the maternally inherited allele, resulting in complete loss of UBE3A expression in neurons.

A continuing need in the art exists for new and effective treatments for AS.

### SUMMARY OF THE INVENTION

In one embodiment, provided herein is an expression cassette comprising a nucleic acid sequence encoding one or more elements of a gene editing system that targets UBE3A-ATS (UBE3A antisense transcript) on a paternal allele in a neuron of a patient having Angelman syndrome and regulatory elements that direct expression thereof in a target cell. Editing of UBE3A-ATS results in unsilencing of the paternal UBE3A allele and permits expression of the UBE3A gene product. The gene editing system may be CRISPR/Cas, a meganuclease, a zinc-finger nuclease, or a TALEN. In certain embodiments, the expression cassette encodes a CRISPR-associated nuclease, optionally Cas9 (e.g., SaCas9), and an sgRNA having a sequence that specifically binds a UBE3A-ATS target sequence. In certain embodiments, the sgRNA comprises any of SEQ ID NOs: 1-32. In certain embodiments, the UBE3A-ATS target sequence is downstream of the UBE3A 3′UTR. In further embodiments, the target sequence is located at chr15: 25,278,409-25,333,728 (hg38 genome assembly) and/or in a sequence of UBE3A-ATS complementary to the region between the UBE3A 3′UTR and SNORD109B ORF on chromosome 15.

An expression cassette provided herein may be included in a non-viral or viral vector. In certain embodiments, the viral vector is an adeno-associated virus (AAV), bocavirus, an adenovirus, a lentivirus, or a retrovirus.

In one embodiment, provided herein is a recombinant adeno-associated virus (rAAV) useful as a CNS-directed therapeutic for treatment of Angelman syndrome (AS). The rAAV comprises an AAV capsid, and a vector genome packaged therein, where the vector genome comprises: (a) an AAV 5′ inverted terminal repeat (ITR); (b) a nucleic acid sequence encoding one or more elements of a gene editing system that targets UBE3A-ATS; (c) regulatory elements that direct expression of the one or more elements of the gene editing system; and (d) an AAV 3′ ITR. In certain embodiments, the gene targeting system comprises a CRISPR endonuclease and a sgRNA that specifically binds a UBE3A-ATS target sequence. The CRISPR endonuclease may be Cas9, optionally SaCas9. In certain embodiments, the capsid is an AAV9 capsid or variant thereof or an AAVhu68 capsid or variant thereof.

In other embodiments, provided herein is a pharmaceutical composition comprising at least an expression cassette, a vector, or an rAAV for delivery of a gene-editing system described herein and a physiologically compatible carrier, buffer, adjuvant, and/or diluent.

In certain embodiments, provided herein is a method of treating AS by administering to a subject in need thereof an expression cassette, a vector, or a rAAV to deliver a gene-editing system, wherein editing of UBE3A-ATS results in enhanced expression of UBE3A from a paternal allele in a neuron. Also provided is a method for treating one or more symptoms of Angelman syndrome (AS) in a patient having deficient UBE3A expression in neurons, wherein the method comprises delivering a nucleic acid sequence that encodes one or more elements of a gene editing system that targets a sequence in UBE3A-ATS downstream of the UBE3A 3′UTR to modify the UBE3A-ATS coding sequence. Editing of UBE3A-ATS results in unsilencing UBE3A expression on a paternal allele of a patient having a deficiency in UBE3A expression from a maternal allele and provides for expression of the UBE3A gene product from the paternal allele. In certain embodiments, the method provides for improve symptoms of Angelman disease, including one or more of delayed development, intellectual disability, severe speech impairment, ataxia and/or epilepsy.

In certain embodiments, provided herein is an expression cassette, vector, rAAV, pharmaceutical for use in treating a patient having Angelman syndrome (AS).

Other aspects and advantages of these methods and compositions are described further in the following detailed description.

### BRIEF DESCRIPTION OF THE FIGURES

FIG. 1 shows an overview of a strategy to unsilence the paternal Ube3a allele. Ube3a shows bi-allelic expression in healthy cells but mono-allelic expression in healthy neurons, where UBE3A-ATS inhibits paternal UBE3A expression. AS subjects lack the maternal Ube3a locus, which prevents UBE3A expression in neurons. Interference with UBE3A-ATS leads to paternal UBE3A allele expression, thus restoring UBE3A protein expression in neurons.

FIG. 2A-FIG. 2I show in vivo gene editing of Ube3α-ATS causes indel formation and expression of Ube3α-YFP reporter. (FIG. 2A) Schematic mouse Ube3a genomic locus [adapted from Meng L et al. Nature. 2015; 518(7539):409-12]. The region targeted in this study by sgRNAs is indicated. IC, imprinting center; 3′UTR, 3′ untranslated region; snoRNA, small-nucleolar RNA. (FIG. 2B) In vitro indel frequencies for screened sgRNAs. (FIG. 2C) Ube3α^{m+/pYFP} mice were injected with ATS-GE vector at indicted timepoints. After three weeks, Amplicon-Seq with cortical samples revealed an average of 14.7% of cells with indels in neonatal injected pups; the indel frequency was <2.9% at all other time points. Non-targeting CRISRP/Cas9 or CRISPR/dCas9 resulted in indel formation in <0.2% (one-way ANOVA with Tukey's pairwise comparison, 2-6 mice/group). (FIG. 2D) Indels persisted in Ube3α^{m+/pYFP} mice neonatal-injected with 1×10¹¹ gc ATS-GE vector (one-way ANOVA with Tukey's pairwise comparison, 3-6 mice/group). (FIG. 2E, FIG. 2F) Representative Western blots for cortices from (FIG. 2C) demonstrate robust expression of paternal Ube3α-YFP when probed with YFP antibodies (FIG. 2E) or Ube3a antibodies (FIG. 2F). Relative quantifications normalized to actin are shown below each lane, green arrow in (FIG. 2F) demarcates the quantified Ube3α-YFP bands. NT, non-targeting. (FIG. 2G) Representative immunofluorescence staining for cortices from (FIG. 2C) shows Ube3α-YFP expression in neurons throughout the cortex (scale bar=100 um). (FIG. 2H) qPCR gene expression analysis with primers specific for the Ube3α-ATS transcript. We detected a significant 28% reduction in Ube3α-ATS expression for Ube3α^{m+/pYFP} gene-edited cortex samples (3-15 mice/group, one-way ANOVA with Tukey's pairwise comparison). (FIG. 2I) qPCR gene expression analysis with primers specific for neighboring transcripts detected no differences in expression (3-4 mice/group, One-way ANOVA with Tukey's pairwise comparison, p>0.4). Means are shown with standard error; * p<0.05, *** p<0.001, **** p<0.0001.

FIG. 3A-FIG. 3G show in vivo gene editing of Ube3α-ATS in a Ube3α-KO mouse model. (FIG. 3A) Brains of Ube3α^{m-/p+} mice injected with 1×10¹¹ gc ATS-GE vector were harvested four months later. We detected persistent paternal Ube3a expression in the cerebral cortex by Western blotting with Ube3a antibodies. Relative quantifications of the respective Ube3a band normalized to actin are shown below each lane. (FIG. 3B) Immunohistochemistry (IHC) staining of the brains from FIG. 3A with Ube3a antibodies shows paternal Ubea3a expression throughout the brain. A representative cortical section is shown here (scale bar: 1 mm). Magnified cortical IHC images from Ube3α^{m+/p+} (FIG. 3C), Ube3a^{m-/p+} (FIG. 3D), and gene-edited Ube3a^{m-/p+} (FIG. 3E) cortex (scale bar: 10 μm). (FIG. 3F) Amplicon-Seq analysis from the same cohort as shown in FIG. 3A revealed an average of 19.4% of cells with indels in injected pups. Injection of non-targeting CRISRP/Cas9 resulted in indel formation of 0.2% (5 mice/group). (FIG. 3G) RNA extracted from cortices of the same cohort as shown in FIG. 3A was used to quantify Ube3α-ATS transcript levels at different locations between the site targeted by gene editing (E) and the imprinting center (IC). We observed a significant reduction of Ube3α-ATS starting approximately 5 kb away from E (one-way ANOVA with Tukey's pairwise comparison, 5 mice/group). Means are shown with standard error; * p<0.05, ** p<0.01.

FIG. 4A-FIG. 4E show phenotypic improvement in an AS mouse model after gene editing. Ube3α^{m-/p+} and Ube3α^{m+/p+} littermates received a neonatal injection of 1×10¹¹ gc ATS-GE or control vector. (FIG. 4A) Mouse weight increased over the observation period but showed no group effect [F(1.311, 64.25)=385.3, p>0.2, 15 mice/group]. (FIG. 4B) At 8 weeks of age, we tested motor function with a rotarod apparatus over three consecutive days. On days 2 and 3, gene-edited Ube3α^{m+/p+} mice showed a significant motor improvement compared to Ube3α^{m+/p+} mice that received control vector (two-way ANOVA with Tukey's pairwise comparison, 15-24 mice/group). (FIG. 4C) Gene-edited Ube3α^{m+/p+} mice demonstrated a significant improvement in burying activity compared to Ube3α^{m+/p+} mice that received control vector (one-way ANOVA with Tukey's pairwise comparison, 15-24 mice/group). (FIG. 4D) Gene-edited Ube3α^{m+/p+} mice demonstrated a significant improvement in nest-building skills and activity compared to Ube3α^{m+/p+} mice that received control vector (one-way ANOVA with Tukey's pairwise comparison, 15-24 mice/group). (FIG. 4E) We assessed the same mouse cohort in an open-field arena to determine ambulatory activity. The performance of the gene-edited Ube3α^{m+/p+} mice showed a trend of improvement at all timepoints compared with Ube3α^{m+/p+} mice that had received the control vector (15-24 mice/group, two-way ANOVA: treatment group effect [F (3, 77)=13.48, p<0.001); Tukey's pairwise comparison, p>0.4). Means are shown with standard error, * p<0.05, ** p<0.01.

FIG. 5A-FIG. 5D shows in vivo gene editing of Ube3α-ATS causes indel formation and expression of Ube3a. (FIG. 5A) We quantified vector genomes in brains from Ube3α^{m+/pYFP} mice treated with an AAV-PHP.B vector encoding CRISPR/Cas9, which was either injected at birth (day 0) into the lateral brain ventricles (ICV) or IV injected at an age of 14, 21, or 28 days. The vector genome copies per diploid genome in the cerebral cortex were 12- to 59-fold higher for ICV injection compared with IV injection at later time points (three mice per group, one-way ANOVA with Tukey's pairwise comparison, ** p<0.001). (FIG. 5B) Ube3α^{m+/pYFP} (paternal Ube3α-YFP) mice were injected with an AAV vector encoding CRISPR/Cas9 at birth (day 0), and the cerebral cortices were harvested 21 days later. We performed molecular analysis by AMP-seq to determine the rate of base-pair insertions, deletions, and ITR integrations, which amounted in total to a mean of 12.8%. By comparison, a nontargeted CRISPR/Cas9 construct had an overall rate of 0.4% in the same experiment. Insertion, deletions and integrations were each significantly increased compared to the NT control (three mice per group, two-way ANOVA with Sidak's pairwise comparison, p>0.001). (FIG. 5C), (FIG. 5D) Ube3α^{m+/pYFP} mice were injected with an AAV vector encoding CRISPR/dCas9 (nuclease-deficient Cas9) at birth (day 0), and the cerebral cortices were harvested 21 days later. (FIG. 5C) We did not detect any paternal UBE3A-YFP protein by Western blot with UBE3A antibodies (Ube3α-YFP bands demarcated by green arrow, relative quantity of each Ube3α-YFP band normalize to actin is annotated under each lane); (FIG. 5D) We did not detect any paternal Ube3α-YFP protein by immunofluorescence staining with GFP antibodies (representative images from cortex, scale bar: 100 μm).

FIG. 6A-FIG. 6C shows in vivo gene editing of Ube3α-ATS leads to expression of Ube3a. Brains were harvested from Ube3α-ko mice injected neonatal with ATS-GE vector or untreated wildtype littermates at age 4 months, fixed and processed for immunohistochemistry with Ube3a antibodies. (FIG. 6A and FIG. 6B) Sagittal overview sections. (FIG. 6C) magnifications of FIG. 6B of the annotated brain regions (scale bars: (FIG. 6A), FIG. 6B)— 3 mm; (FIG. 6C)— 300 um)

FIG. 7A-FIG. 7C shows in vivo gene editing of Ube3α-ATS in AS mouse model Ube3α^{m+/p+} mice were injected with an AAV vector encoding CRISPR/dCas9 (nuclease-deficient Cas9) at birth (day 0), and the cerebral cortices were harvested 4 months later. (FIG. 7A) We did not detect any significant paternal Ube3a protein expression by Western blot with Ube3a antibodies (relative quantity normalized to actin annotated to each lane). (FIG. 7B) Likewise, immunohistochemistry staining with Ube3a antibodies did not show any Ube3aexpression (representative images, scale bar: 500 μm). (FIG. 7C) Ube3α^{m+/p+} mice were injected with an AAV vector encoding CRISPR/Cas9 with a targeted or non-targeted (NT) sgRNA at birth (day 0), and the cerebral cortices were harvested four months later. AMP-seq was used to quantify frequency of deletions, insertions or ITR integrations, which amounted to 22% for the edited and 0.5% for the control (NT) group. Insertion, deletions, and integrations were each significantly increased (five mice per group, two-way ANOVA with Sidak's pairwise comparison, p>0.001).

FIG. 8 shows an AAV vector genome and results from screening of sgRNAs for efficiency in targeting the Ube3α-ATS coding region downstream of Ube3a in vitro.

FIG. 9 provides a list of sgRNA sequences and their target locations in a region of human UBE3A-ATS (SEQ ID NOs: 1-32, top to bottom).

### DETAILED DESCRIPTION OF THE INVENTION

The methods and compositions described herein are useful for the treatment of Angelman syndrome (AS), a condition that results from a deletion or mutation in a maternal Ube3a allele and a lack of UBE3A expression in neurons.

The loss of UBE3A expression in AS patients is the result of a combination of a mutation, defect, in the maternally inherited UBE3A allele and silencing of the paternally inherited UBE3A allele, resulting in complete loss of UBE3A expression in neurons. One approach to reinstate UBE3A expression in neurons is to unsilence the paternal UBE3A gene that is fully functional but not expressing.

Paternal UBE3A-silencing is achieved by expression of an antisense transcript (ATS) that is thought to suppress extension of UBE3A mRNA past the transcriptional start site. As demonstrated herein, interfering with the extension of the ATS into the UBE3A coding region allows for full length extension of UBE3A mRNA and protein expression. We administered an adeno-associated virus (AAV) vector providing a CRISPR/Cas9 gene editing system to AS model mice and achieved indel formation in the ATS sequence in up to 21% of mouse neurons in vivo. Indel formation resulted in unsilencing of a paternal Ube3a allele and subsequent protein expression. Expression of Ube3a from the maternal allele was not affected. Further, Ube3α-ATS gene editing in mice selectively reduced the abundance of full-length Ube3α-ATS transcript without unsilencing other genes regulated by Ube3α-ATS (including Snrpn, Snord115, Snord116). Following treatment, UBE3A protein expression in AS mice persisted for at least three months. Treated AS model mice also had improved performance in a neurobehavior test battery. The findings demonstrate that reactivation of Ube3a by gene editing in a limited number of neurons is sufficient to improve disease symptoms in an AS mouse model. Current treatments for AS are symptomatic, including pharmaceutical treatments for seizures and behavioral aspects of the disease. Compared to other approaches that would require periodic re-administration, a gene editing approach for treatment of AS has the potential to be a long-lasting therapy.

In one embodiment, the compositions and methods described herein involve expression cassettes, vectors, and recombinant viruses for delivery of a gene-editing system for treatment of AS.

As used herein, “disease”, “disorder”, and “condition” are used interchangeably, to indicate an abnormal state in a subject. In one embodiment, the disease is Angelman syndrome (AS).

“Patient” or “subject”, as used herein interchangeably, means a male or female mammalian animal, including a human, a veterinary or farm animal, a domestic animal or pet, and animals normally used for clinical research. In one embodiment, the subject of these methods and compositions is a human patient. In one embodiment, the subject of these methods and compositions is a male or female human.

As used throughout this specification and the claims, the terms “comprising”, “containing”, “including”, and its variants are inclusive of other components, elements, integers, steps and the like. Conversely, the term “consisting” and its variants are exclusive of other components, elements, integers, steps and the like.

It is to be noted that the term “a” or “an”, refers to one or more, for example, “a neuron”, is understood to represent one or more neuron(s). As such, the terms “a” (or “an”), “one or more,” and “at least one” is used interchangeably herein.

As used herein, the term “about” means a variability of plus or minus 10% from the reference given, unless otherwise specified.

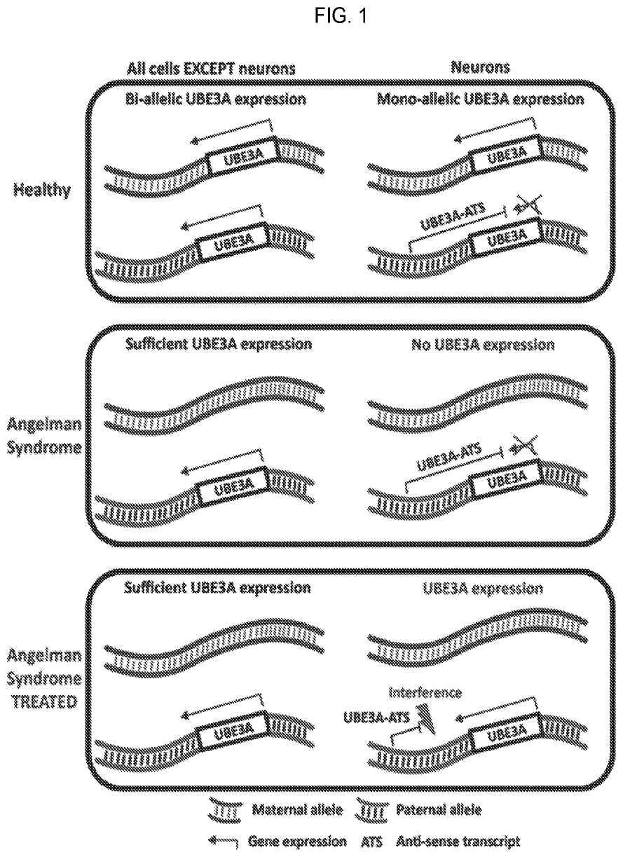

As used herein “UBE3A-ATS” refers to UBE3A antisense transcript. In humans, UBE3A-ATS is also known as small nucleolar RNA host gene 14 (SNHG14); NCBI Gene ID: 104472715, NCBI Reference Sequence: NR 146177.1) (see, e.g., Runte M., et al. Hum. Mol. Genet. 2001; 10:2687-2700, which is incorporated herein by reference). Without wishing to be bound by theory, UBE3A-ATS extends into the UBE3A gene on the paternal chromosome in neuronal cells and interferes with transcription of UBE3A. In non-neuronal cells, transcription of UBE3A-ATS does not extend to UBE3A and UBE3A remains biallelically expressed (see FIG. 1). Mouse and human UBE3A-ATS are located on different chromosomes (7 and 15, respectively); however the transcript is located in a region (known as the Prader-Willi syndrome (PWS)/Angelman syndrome (AS) region) that is highly conserved between mouse and human. As described herein, in certain embodiments, the target sequence for gene editing is in human UBE3A-ATS in a region downstream of the UBE3A 3′UTR. In certain embodiments, the target sequence in human UBE3A-ATS is located at chr15: 25,278,409-25,333,728 (hg38 genome assembly). In yet another embodiment, the target sequence in human UBE3A-ATS in a region between the 3′ UTR of UBE3A and SNORD109B (NCBI Reference Sequence: NR 001289.1). In one aspect, provided herein are compositions and methods for editing UBE3A-ATS in a manner that enhances UBE3A expression of a paternal allele without altering expression of other genes regulated by Ube3α-ATS. In certain embodiments, editing of human Ube3α-ATS does not alter expression of SNORD109B.

Nucleic acid sequences described herein can be cloned using routine molecular biology techniques, or generated de novo by DNA synthesis, which can be performed using routine procedures by service companies having business in the field of DNA synthesis and/or molecular cloning (e.g. GeneArt, GenScript, Life Technologies, Eurofins). The nucleic acid sequences encoding aspects of a UBE3A-ATS editing system described herein are assembled and placed into any suitable genetic element, e.g., naked DNA, phage, transposon, cosmid, episome, etc., which transfers the sequences carried thereon to a host cell, e.g., for generating non-viral delivery systems (e.g., RNA-based systems, naked DNA, or the like), or for generating viral vectors in a packaging host cell, and/or for delivery to a host cells in a subject. In one embodiment, the genetic element is a vector. In one embodiment, the genetic element is a plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, N.Y. (2012).

### Expression Cassettes

As used herein, an “expression cassette” refers to a nucleic acid molecule which encodes one or more elements of a gene editing system, e.g. an endonuclease and targeting sequence (e.g. crRNA sequence of a CRISPR/Cas system). An expression cassette also contains a promoter and may contain additional regulatory elements that control expression of the gene editing system in a host cell. In one embodiment, the expression cassette may be packaged into the capsid of a viral vector (e.g., a viral particle). In one embodiment, such an expression cassette for generating a viral vector as described herein is flanked by packaging signals of the viral genome and other expression control sequences such as those described herein. For example, for an AAV viral vector, the packaging signals are a 5′ AAV inverted terminal repeat (ITR) and a 3′ AAV ITR.

As used herein, the term “operably linked” or “operatively associated” refers to both expression control sequences or regulatory elements that are contiguous with the gene of interest and expression control sequences that act in trans or at a distance to control the gene of interest.

As described herein, regulatory elements comprise but not limited to: promoter; enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence).

In one embodiment, the expression cassette comprises regulatory elements that direct expression of a sequence encoding one or more elements of a gene editing system for targeting UBE3A-ATS. In one embodiment, the regulatory elements comprise one or more promoters. In certain embodiments, the expression cassette includes a CMV promoter. In certain embodiments, the promoter is a neuron specific promoter. In certain embodiments, a suitable promoter may include without limitation, an elongation factor 1 alpha (EF1 alpha) promoter (see, e.g., Kim D W et al, Use of the human elongation factor 1 alpha promoter as a versatile and efficient expression system. Gene. 1990 Jul. 16; 91(2):217-23), a Synapsin 1 promoter (see, e.g., Kugler S et al, Human synapsin 1 gene promoter confers highly neuron-specific long-term transgene expression from an adenoviral vector in the adult rat brain depending on the transduced area. Gene Ther. 2003 February; 10(4):337-47), a neuron-specific enolase (NSE) promoter (see, e.g., Kim J et al, Involvement of cholesterol-rich lipid rafts in interleukin-6-induced neuroendocrine differentiation of LNCaP prostate cancer cells. Endocrinology. 2004 February; 145(2):613-9. Epub 2003 Oct. 16), or a CB6 promoter (see, e.g., Large-Scale Production of Adeno-Associated Viral Vector Serotype-9 Carrying the Human Survival Motor Neuron Gene, Mol Biotechnol. 2016 January; 58(1):30-6. doi: 10.1007/s12033-015-9899-5). Other suitable promoters include CAG promoter, which comprises (C) the cytomegalovirus (CMV) early enhancer element, (A) the promoter, the first exon and the first intron of chicken beta-actin gene, and (G) the splice acceptor of the rabbit beta-globin gene. See, e.g., Alexopoulou, Annika N., et al. BMC cell biology 9.1 (2008): 2. Although less desired, other promoters, such as viral promoters, constitutive promoters, inducible promoters, regulatable promoters (see, e.g., WO 2011/126808 and WO 2013/04943), or a promoter responsive to physiologic cues may be used may be utilized in the vectors described herein. In certain embodiments, the expression cassette includes an U6 promoter. In another embodiment, the regulatory elements comprise an enhancer. In a further embodiment, the enhancer(s) is selected from one or more of an APB enhancer, an ABPS enhancer, an alpha mic/bik enhancer, a TTR enhancer, an en34 enhancer, an ApoE enhancer, a CMV enhancer, or an RSV enhancer. In yet another embodiment, the regulatory elements comprise an intron. In a further embodiment, the intron is selected from CBA, human beta globin, IVS2, SV40, bGH, alpha-globulin, beta-globulin, collagen, ovalbumin, or p53. In one embodiment, the regulatory elements comprise a polyA. In a further embodiment, the polyA is a synthetic polyA or from bovine growth hormone (bGH), human growth hormone (hGH), SV40, rabbit β-globin (RGB), or modified RGB (mRGB). In another embodiment, the regulatory elements may comprise a WPRE sequence. In yet another embodiment, the regulatory elements comprise a Kozak sequence.

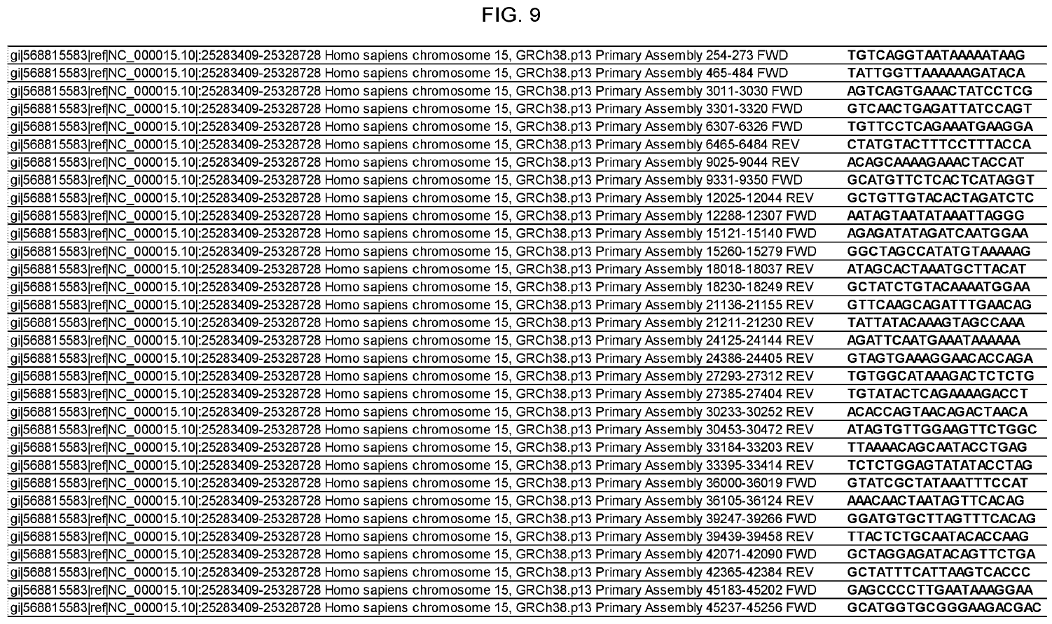

In certain embodiments, an expression cassette is provided that includes a U6 promoter operably linked to sequence encoding a sgRNA. In certain embodiments, the expression cassette includes at a minimum a U6 promoter operably linked to a sgRNA coding sequence and a neuron specific promoter (e.g. human synapsin promoter) operably linked to a Cas9 coding sequence. An exemplary vector genome is depicted in FIG. 9.

The term “expression” is used herein in its broadest meaning and comprises the production of RNA, of protein, or of both RNA and protein. With respect to RNA, the term “expression” or “translation” relates in particular to the production of peptides or proteins. Expression may be transient or may be stable.

Expression cassettes can be delivered via any suitable delivery system. Suitable non-viral delivery systems are known in the art (see, e.g., Ramamoorth and Narvekar. J Clin Diagn Res. 2015 January; 9(1):GE01-GE06, which is incorporated herein by reference) and can be readily selected by one of skill in the art and may include, e.g., naked DNA, naked RNA, dendrimers, PLGA, polymethacrylate, an inorganic particle, a lipid particle (e.g., a lipid nanoparticle or LNP), or a chitosan-based formulation.

In one embodiment, the vector is a non-viral plasmid that comprises an expression cassette described thereof, e.g., “naked DNA”, “naked plasmid DNA”, RNA, and mRNA; coupled with various compositions and nano particles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference.

Provided herein are compositions comprising a nucleic acid sequence encoding one or more elements of a gene editing system and methods of use thereof for editing UBE3A-ATS. As used herein, “gene editing system” refers to technologies or molecular machinery for modifying genetic material, typically with specificity for a particular gene or nucleic acid sequence (including, e.g., target sequences or motifs). Such gene editing systems are designed to modify a target site in the genome or introduce a mutation. As used herein, a “mutation” or “modification”, unless otherwise stated, can refer to any alteration of a genomic sequence, including but not limited to small nucleotide insertions or deletions (indels) or a larger deletion, insertion, or inversion. In certain embodiments, the introduction a mutation or modification is referred to as “editing” or “gene editing”.

Terms such as “target site” and “target sequence”, unless indicated otherwise, are used herein to refer to a sequence that is recognized by one or more elements of a gene-editing system. For example, a sgRNA includes a sequence that binds (i.e. is complementary to) a target site or target sequence in the genome.

In certain embodiments, the gene editing system is a Clustered Regulatory Interspaced Short Palindromic Repeats (CRISPR) system for modifying UBE3A-ATS. In one embodiment, provided herein is a CRISPR/Cas dual vector system (see, e.g. WO 2016/176191, which is incorporated herein by reference). Alternatively, in certain embodiments, a suitable gene editing system includes a zinc-finger nuclease (ZFN) to induce DNA double-strand breaks, which may or may not be in conjunction with delivery of an exogenous DNA donor substrate (See, e.g., Ellis et al, Gene Therapy (epub January 2012) 20:35-42 which is incorporated herein by reference). In other embodiments, a suitable gene editing system includes a meganuclease (see, e.g., in U.S. Pat. Nos. 8,445,251; 9,340,777; 9,434,931; 9,683,257, and WO 2018/195449, each of which is incorporated herein by reference) or transcription activator-like (TAL) effector nucleases (TALENs).

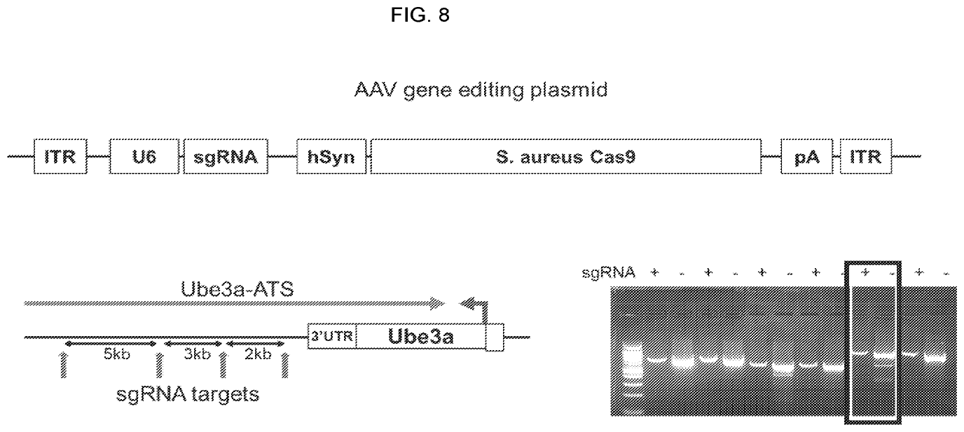

In certain embodiments, a suitable CRISPR gene editing system includes, at a minimum, a Cas9 enzyme and a sgRNA specific for a target site in the Ube3α-ATS coding sequence. Accordingly, in one embodiment, the gene editing vector comprises a Cas9 gene as the editing enzyme and an sgRNA which is at least 20 nucleotides in length and specifically binds to a selected site in Ube3α-ATS 5′ to a protospacer-adjacent motif (PAM) which is specifically recognized by the Cas9. In certain embodiment, the expression cassette or vector genome includes a nucleic acid sequence encoding the sgRNA molecule and a nucleic acid sequence encoding a Cas9 enzyme (see, e.g. FIG. 8). In certain embodiments, the gene editing system also includes a donor or repair template. The expression cassette providing the donor template may be the same as the expression cassettes encoding the sgRNA and Cas9, or a different expression cassette. Thus, in certain embodiments, a dual-vector system (as described for example in WO 2016/176191) is provided, wherein the gene editing system includes an expression cassette comprising a Cas9 gene under control of regulatory sequences which direct its expression and a second expression cassette comprising a sgRNA and a donor template.

“Cas9” (CRISPR associated protein 9) refers to family of RNA-guided DNA endonucleases which is characterized by two signature nuclease domains, RuvC (cleaves non-coding strand) and HNH (coding strand). Suitable bacterial sources of Cas9 include Staphylococcus aureus (SaCas9), Stapylococcus pyogenes (SpCas9), and Neisseria meningitides (K M Estelt et al, Nat Meth, 10:1116-21 (2013)). The wild-type coding sequences may be utilized in the constructs described herein. Alternatively, bacterial codons are optimized for expression in humans, e.g. using any of a variety of known human codon optimizing algorithms. Other endonucleases with similar properties may optionally be substituted. See, e.g., the public CRISPR database (db) accessible at crispr.u-psud.fr/crispr. CRISPR/Cas9 gene targeting requires a single guide RNA (sgRNA) that contains a targeting sequence (crRNA sequence) and a Cas9 nuclease-recruiting sequence (tracrRNA). The crRNA region is a 20-nucleotide sequence that is homologous to a target site and will direct Cas9 nuclease activity. Strategies for identifying suitable target sites in the genome while also eliminating off target effects are known to those of skill in the art (see, e.g., ChopChop available online at chopchop.cbu.uib.no/). Provided in the table below are sequences for the design of sgRNA suitable for use in a SaCas9/CRISPR gene editing system for targeting human UBE3A-ATS, as described herein. In certain embodiments, the expression cassette comprises a sequence encoding an sgRNA comprising any of SEQ ID NOs: 1-32.

| Targeting Sequence | SEQ ID NO: |
|---|---|
| TGTCAGGTAATAAAAATAAG | 1 |
| TATTGGTTAAAAAAGATACA | 2 |
| AGTCAGTGAAACTATCCTCG | 3 |
| GTCAACTGAGATTATCCAGT | 4 |
| TGTTCCTCAGAAATGAAGGA | 5 |
| CTATGTACTTTCCTTTACCA | 6 |
| ACAGCAAAAGAAACTACCAT | 7 |
| GCATGTTCTCACTCATAGGT | 8 |
| GCTGTTGTACACTAGATCTC | 9 |
| AATAGTAATATAAATTAGGG | 10 |
| AGAGATATAGATCAATGGAA | 11 |
| GGCTAGCCATATGTAAAAAG | 12 |
| ATAGCACTAAATGCTTACAT | 13 |
| GCTATCTGTACAAAATGGAA | 14 |
| GTTCAAGCAGATTTGAACAG | 15 |
| TATTATACAAAGTAGCCAAA | 16 |
| AGATTCAATGAAATAAAAAA | 17 |
| GTAGTGAAAGGAACACCAGA | 18 |
| TGTGGCATAAAGACTCTCTG | 19 |
| TGTATACTCAGAAAAGACCT | 20 |
| ACACCAGTAACAGACTAACA | 21 |
| ATAGTGTTGGAAGTTCTGGC | 22 |
| TTAAAACAGCAATACCTGAG | 23 |
| TCTCTGGAGTATATACCTAG | 24 |
| GTATCGCTATAAATTTCCAT | 25 |
| AAACAACTAATAGTTCACAG | 26 |
| GGATGTGCTTAGTTTCACAG | 27 |
| TTACTCTGCAATACACCAAG | 28 |
| GCTAGGAGATACAGTTCTGA | 29 |
| GCTATTTCATTAAGTCACCC | 30 |
| GAGCCCCTTGAATAAAGGAA | 31 |
| GCATGGTGCGGGAAGACGAC | 32 |

In another embodiment, the CRISPR gene editing system may be Cpfl (CRISPR from Prevotella and Francisella). Cpfl's preferred PAM is 5′-TTN; this contrasts with that of SpCas9 (5′-NGG) and SaCas9 (5′-NNGRRT; N=any nucleotide; R=adenine or guanine) in both genomic location and GC-content. While at least 16 Cpfl nucleases have been identified, two humanized nucleases (AsCpfl and LbCpfl) are particularly useful. See, www.addgene.org/69982/sequences/#depositor-full (AsCpfl sequences; and www.addgene.org/69988/sequences/#depositor-full (LbCpfl sequences), which are incorporated herein by reference. Further, Cpfl does not require a tracrRNA; allowing use of shorter guide RNAs (about 42 nucleotides) as compared to Cas9. Plasmids may be obtained from Addgene, a public plasmid database.

As described herein, a gene editing system is utilized to introduce a mutation in a paternal Ube3α-ATS allele in target cell. In some embodiments, the target polynucleotide sequence (i.e. a Ube3α-ATS sequence) is cleaved such that a double-strand break results. In some embodiments, the target polynucleotide sequence is cleaved such that a single-strand break results. In certain embodiments, the alteration is an insertion or deletion (indel), which can result in random insertion/deletion mutations at the site of junction as a result of non-homologous end joining. Indel mutations occurring within the coding region of a gene can result in frame-shift and a premature stop codon, and disrupt transcription. Alternatively, a repair template in the form of a plasmid or single-stranded oligodeoxynucleotides (ssODN) can be supplied to leverage the homology-directed repair (HDR) pathway, which allows high fidelity and precise editing.

In certain embodiments, a viral vector is used to deliver one more elements of the gene editing system. While the examples below describe use of AAV vectors and the following discussion focuses on AAV vectors, it will be understood that a different, partially or wholly integrating vector or virus may be used in the system in place of the gene editing vector and/or the vector carrying template. See, e.g., Jinek, M.; Chilynksi, K.; Fonfara, I.; Hauer, M.; Doudna, J.; Charpentier, E., (Aug. 17, 2012). “A programmable dual-RNA-guided DNA endonuclease in adaptive bacterial immunity”. Science. 337 (6069): 816-821. Bibcode:2012 Sci.337.816J. doi:10.1126/science.1225829. PMID 22745249; U.S. Pat. Nos. 8,697,359; 9,909,122, US 2017/0051312; US 2017/0137801; US 2017/0166893; US2017/0360048; US 2018/0002682, which are incorporated by reference in their entirety.

In certain embodiments, the vector delivers one or more components (e.g., the guide RNA and the endonuclease) of the genome editing system, such as CRISPR/Cas9. In another embodiment, a combination or dual AAV vector system is provided to deliver the components of the CRISPR system when co-administered to a subject (see, e.g. WO 2016/176191, which is incorporated by reference herein in its entirety). The vectors may be formulated together or separately and delivered essentially simultaneously, preferably by the same route.

In certain embodiments, one or more mutations may be introduced into a target sequence (e.g., UBE3A-ATS) using a gene editing system described herein. In certain embodiments, a vector is provided to deliver a donor or repair template, which is sequence designed such that when it is introduced into the target sequence there is disruption of transcription of UBE3A-ATS, including e.g., early termination.

A variety of conventional vector elements may be used to enhance gene editing activity in a target cell. For example, a system designed for treatment of to treat AS may be designed such that a CRISPR enzyme is expressed under the control of a neuron-specific promoter (e.g., human synapsin 1).

Optionally, the expression cassette may include miRNA target sequences in the untranslated region(s). The miRNA target sequences are designed to be specifically recognized by miRNA present in cells in which transgene expression is undesirable and/or reduced levels of transgene expression are desired. In certain embodiments, the expression cassette includes miRNA target sequences that specifically reduce expression of the nuclease in dorsal root ganglion (DRG). In certain embodiments, the miRNA target sequences are located in the 3′ UTR, 5′ UTR, and/or in both 3′ and 5′ UTR, In some embodiments, the miRNA target sequences are operably linked to the regulatory sequences in the expression cassette. In certain embodiments, the expression cassette comprises at least two tandem repeats of DRG-specific miRNA target sequences, wherein the at least two tandem repeats comprise at least a first miRNA target sequence and at least a second miRNA target sequence which may be the same or different. In certain embodiments, the tandem miRNA target sequences are continuous or are separated by a spacer of 1 to 10 nucleic acids, wherein said spacer is not an miRNA target sequence.

In certain embodiments, the vector genome or expression cassette contains at least one miRNA target sequence that is a miR-183 target sequence. In certain embodiments, the vector genome or expression cassette contains an miR-183 target sequence that includes AGTGAATTCTACCAGTGCCATA (SEQ ID NO: 33), where the sequence complementary to the miR-183 seed sequence is underlined. In certain embodiments, the vector genome or expression cassette contains more than one copy (e.g. two or three copies) of a sequence that is 100% complementary to the miR-183 seed sequence. In certain embodiments, a miR-183 target sequence is about 7 nucleotides to about 28 nucleotides in length and includes at least one region that is at least 100% complementary to the miR-183 seed sequence. In certain embodiments, a miR-183 target sequence contains a sequence with partial complementarity to SEQ ID NO: 33 and, thus, when aligned to SEQ ID NO: 33, there are one or more mismatches. In certain embodiments, a miR-183 target sequence comprises a sequence having at least 1, 2, 3, 4, 5, 6, 7, 8, 9, or 10 mismatches when aligned to SEQ ID NO: 33, where the mismatches may be non-contiguous. In certain embodiments, a miR-183 target sequence includes a region of 100% complementarity which also comprises at least 30% of the length of the miR-183 target sequence. In certain embodiments, the region of 100% complementarity includes a sequence with 100% complementarity to the miR-183 seed sequence. In certain embodiments, the remainder of a miR-183 target sequence has at least about 80% to about 99% complementarity to miR-183. In certain embodiments, the expression cassette or vector genome includes a miR-183 target sequence that comprises a truncated SEQ ID NO: 33, i.e., a sequence that lacks at least 1, 2, 3, 4, 5, 6, 7, 8, 9, or 10 nucleotides at either or both the 5′ or 3′ ends of SEQ ID NO: 33. In certain embodiments, the expression cassette or vector genome comprises a transgene and one miR-183 target sequence. In yet other embodiments, the expression cassette or vector genome comprises at least two, three or four miR-183 target sequences.

In certain embodiments, the vector genome or expression cassette contains at least one miRNA target sequence that is a miR-182 target sequence. In certain embodiments, the vector genome or expression cassette contains an miR-182 target sequence that includes AGTGTGAGTTCTACCATTGCCAAA (SEQ ID NO: 34). In certain embodiments, the vector genome or expression cassette contains more than one copy (e.g. two or three copies) of a sequence that is 100% complementary to the miR-182 seed sequence. In certain embodiments, a miR-182 target sequence is about 7 nucleotides to about 28 nucleotides in length and includes at least one region that is at least 100% complementary to the miR-182 seed sequence. In certain embodiments, a miR-182 target sequence contains a sequence with partial complementarity to SEQ ID NO: 34 and, thus, when aligned to SEQ ID NO: 34, there are one or more mismatches. In certain embodiments, a miR-183 target sequence comprises a sequence having at least 1, 2, 3, 4, 5, 6, 7, 8, 9, or 10 mismatches when aligned to SEQ ID NO: 34, where the mismatches may be non-contiguous. In certain embodiments, a miR-182 target sequence includes a region of 100% complementarity which also comprises at least 30% of the length of the to miR-182 target sequence. In certain embodiments, the region of 100% complementarity includes a sequence with 100% complementarity to the miR-182 seed sequence. In certain embodiments, the remainder of a miR-182 target sequence has at least about 80% to about 99% complementarity to miR-182. In certain embodiments, the expression cassette or vector genome includes a miR-182 target sequence that comprises a truncated SEQ ID NO: 34, i.e., a sequence that lacks at least 1, 2, 3, 4, 5, 6, 7, 8, 9, or 10 nucleotides at either or both the 5′ or 3′ ends of SEQ ID NO: 34. In certain embodiments, the expression cassette or vector genome comprises a transgene and one miR-182 target sequence. In yet other embodiments, the expression cassette or vector genome comprises at least two, three or four miR-182 target sequences.

The term “tandem repeats” is used herein to refer to the presence of two or more consecutive miRNA target sequences. These miRNA target sequences may be continuous, i.e., located directly after one another such that the 3′ end of one is directly upstream of the 5′ end of the next with no intervening sequences, or vice versa. In another embodiment, two or more of the miRNA target sequences are separated by a short spacer sequence.

As used herein, as “spacer” is any selected nucleic acid sequence, e.g., of 1, 2, 3, 4, 5, 6, 7, 8, 9 or 10 nucleotides in length which is located between two or more consecutive miRNA target sequences. In certain embodiments, the spacer is 1 to 8 nucleotides in length, 2 to 7 nucleotides in length, 3 to 6 nucleotides in length, four nucleotides in length, 4 to 9 nucleotides, 3 to 7 nucleotides, or values which are longer. Suitably, a spacer is a non-coding sequence. In certain embodiments, the spacer may be of four (4) nucleotides. In certain embodiments, the spacer is GGAT. In certain embodiments, the spacer is six (6) nucleotides. In certain embodiments, the spacer is CACGTG or GCATGC.

In certain embodiments, the tandem repeats contain two, three, four or more of the same miRNA target sequence. In certain embodiments, the tandem repeats contain at least two different miRNA target sequences, at least three different miRNA target sequences, or at least four different miRNA target sequences, etc. In certain embodiments, the tandem repeats may contain two or three of the same miRNA target sequence and a fourth miRNA target sequence which is different.

In certain embodiments, there may be at least two different sets of tandem repeats in the expression cassette. For example, a 3′ UTR may contain a tandem repeat immediately downstream of the transgene, UTR sequences, and two or more tandem repeats closer to the 3′ end of the UTR. In another example, the 5′ UTR may contain one, two or more miRNA target sequences. In another example the 3′ may contain tandem repeats and the 5′ UTR may contain at least one miRNA target sequence.

In certain embodiments, the expression cassette contains two, three, four or more tandem repeats which start within about 0 to 20 nucleotides of the stop codon for the transgene. In other embodiments, the expression cassette contains the miRNA tandem repeats at least 100 to about 4000 nucleotides from the stop codon for the transgene. See, PCT/US19/67872, filed Dec. 20, 2019, which is incorporated by reference herein and which claims priority to US Provisional U.S. Patent Application No. 62/783,956, filed Dec. 21, 2018, which is hereby incorporated by reference.

It should be understood that the compositions in the expression cassettes described herein are intended to be applied to the compositions and methods described across the Specification.

### Vectors

In certain embodiments, one or more elements of gene editing system are encoded by nucleic acid sequence that is delivered to neurons by a vector or a viral vector, of which many are known and available in the art. In one embodiment, provided is a vector comprising the UBE3A-ATS targeting gene editing system as described herein. In one embodiment, provided is a vector comprising an expression cassette as described herein. In one embodiment, the vector is a non-viral vector. In a further embodiment, the non-viral vector is a plasmid. In another embodiment, the vector is a viral vector. Viral vectors include any virus suitable for gene therapy, including but not limited to a bocavirus, adenovirus, adeno-associated virus (AAV), herpes virus, lentivirus, retrovirus, or parvovirus. However, for ease of understanding, the adeno-associated virus is referenced herein as an exemplary virus vector. Thus, in one embodiment, an adeno-associated viral vector comprising a nucleic acid sequence one or more elements of gene editing system operatively linked to regulatory elements therefor is provided.

A “vector” as used herein is a biological or chemical moiety comprising a nucleic acid sequence which can be introduced into an appropriate target cell for replication or expression of a nucleic acid sequence. Examples of a vector include but are not limited to a recombinant virus, a plasmid, Lipoplexes, a Polymersome, Polyplexes, a dendrimer, a cell penetrating peptide (CPP) conjugate, a magnetic particle, or a nanoparticle. In one embodiment, a vector is a nucleic acid molecule having an exogenous or heterologous engineered nucleic acid encoding a functional gene product, which can then be introduced into an appropriate target cell. Such vectors preferably have one or more origins of replication, and one or more site into which the recombinant DNA can be inserted. Vectors often have means by which cells with vectors can be selected from those without, e.g., they encode drug resistance genes. Common vectors include plasmids, viral genomes, and “artificial chromosomes”. Conventional methods of generation, production, characterization, or quantification of the vectors are available to one of skill in the art.

As used herein, a recombinant viral vector is any suitable viral vector which targets the desired cell(s). Thus, the recombinant viral vectors described herein preferably target one or more of the cells and tissues affected by Angelman syndrome, including cells of the central nervous system (e.g., brain). The examples provide illustrative recombinant adeno-associated viruses (rAAV). However, other suitable viral vectors may include, e.g., a recombinant adenovirus, a recombinant parvovirus such a recombinant bocavirus, a hybrid AAV/bocavirus, a recombinant herpes simplex virus, a recombinant retrovirus, or a recombinant lentivirus. In preferred embodiments, these recombinant viruses are replication-defective.

A “replication-defective” virus or viral vector refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication. Such replication-defective viruses may be adeno-associated viruses (AAV), adenoviruses, lentiviruses (integrating or non-integrating), or another suitable virus source.

“Plasmid” or “plasmid vector” generally is designated herein by a lower-case p preceded and/or followed by a vector name. Plasmids, other cloning and expression vectors, properties thereof, and constructing/manipulating methods thereof that can be used in accordance with the present invention are readily apparent to those of skill in the art. In one embodiment, the elements of a gene editing system as described herein or the expression cassette as described herein are engineered into a suitable genetic element (a vector) useful for generating viral vectors and/or for delivery to a host cell, e.g., naked DNA, phage, transposon, cosmid, episome, etc., which transfers the sequences carried thereon. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

The term “transgene” or “gene of interest” as used interchangeably herein means an exogenous and/or engineered protein-encoding nucleic acid sequence that is under the control of a promoter and/or other regulatory elements in an expression cassette, rAAV genome, recombinant plasmid or production plasmid, vector, or host cell described in this specification.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence with which the protein or nucleic acid in question is not found in the same relationship to each other in nature.

As used herein, the term “host cell” may refer to the packaging cell line in which a vector (e.g., a recombinant AAV) is produced from a production plasmid. In the alternative, the term “host cell” may refer to any target cell in which expression of a gene editing system described herein is desired. Thus, a “host cell,” refers to a prokaryotic or eukaryotic cell that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to cultures of cells of various mammalian species for in vitro assessment of the compositions described herein. In other embodiments herein, the term “host cell” refers to the cells employed to generate and package the viral vector or recombinant virus. Still in other embodiment, the term “host cell” is intended to reference a target cell of the subject being treated in vivo for AS. In a further embodiment, the term “host cell” is a neuron, e.g. a neuron of the CNS.

As used herein, the term “target cell” refers to any target cell in which expression of a heterologous nucleic acid sequence or protein is desired. In certain embodiments, the target cell is a neuron of the CNS, in particular a neuron with a mutated or defective maternal UBE3A allele or a neuron that lacks UBE3A expression.

As used herein, a “vector genome” refers to the nucleic acid sequence packaged inside a viral vector. In one example, a “vector genome” contains, at a minimum, from 5′ to 3′, a vector-specific sequence, a nucleic acid sequence encoding one or more elements of a gene editing system (e.g., a CRISPR/Cas enzyme and sgRNA operably linked to regulatory control sequences which direct their expression in a target cell), where the vector-specific sequence may be a terminal repeat sequence which specifically packages the vector genome into a viral vector capsid or envelope protein. For example, AAV inverted terminal repeats are utilized for packaging into AAV and certain other parvovirus capsids. Lentivirus long terminal repeats may be utilized where packaging into a lentiviral vector is desired. Similarly, other terminal repeats (e.g., a retroviral long terminal repeat), or the like may be selected.

The term “AAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV nuclease (e.g., DNase)-resistant particle having an AAV protein capsid into which is packaged expression cassette flanked by AAV inverted terminal repeat sequences (ITRs) for delivery to target cells. A nuclease-resistant recombinant AAV (rAAV) indicates that the AAV capsid has fully assembled and protects these packaged vector genome sequences from degradation (digestion) during nuclease incubation steps designed to remove contaminating nucleic acids which may be present from the production process. In many instances, the rAAV described herein is DNase resistant.

The source of the AAV capsid may be one of any of the dozens of naturally occurring and available adeno-associated viruses, as well as engineered AAVs. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10). These documents also describe other AAV which may be selected for generating AAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models. Unless otherwise specified, the AAV capsid, ITRs, and other selected AAV components described herein, may be readily selected from among any AAV, including, without limitation, the AAVs commonly identified as AAV1, AAV2, AAV3, AAV4, AAV5, AAV6, AAV7, AAV8, AAV9, AAV8 bp, AAV7M8 and AAVAnc80. See, e.g., WO 2005/033321, which is incorporated herein by reference. In one embodiment, the AAV capsid is an AAV9 capsid or variant thereof. In certain embodiments, the capsid protein is designated by a number or a combination of numbers and letters following the term “AAV” in the name of the rAAV vector. See, also PCT/US19/169004 and PCT/US19/198961, each entitled “Novel Adeno-Associated Virus (AAV) Vectors, AAV Vectors Having Reduced Capsid Deamidation And Uses Therefor”, which are incorporated by reference herein in their entireties.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

As used herein, relating to AAV, the term “variant” means any AAV sequence which is derived from a known AAV sequence, including those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3). In one embodiment, the AAV capsid shares at least 95% identity with the AAV8 vp3. In another embodiment, a self-complementary AAV is used.

The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, Va.). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

As used herein, the terms “rAAV” and “artificial AAV” used interchangeably, mean, without limitation, an AAV comprising a capsid protein and a vector genome packaged therein, wherein the vector genome comprising a nucleic acid heterologous to the AAV. In one embodiment, the capsid protein is a non-naturally occurring capsid. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/5 and AAV2/8 are exemplary pseudotyped vectors. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, N.Y. (2012).

In certain embodiments, the AAV capsid is selected from among natural and engineered clade F adeno-associated viruses. In the examples below, the clade F adeno-associated virus is AAVhu68. See, WO 2018/160582, which is incorporated by reference herein in its entirety. In other embodiments, another AAV capsid is selected from a different clade, e.g., clade A, B, C, D, or E, or from an AAV source outside of any of these clades. For example, another suitable capsid is AAVrh91. See WO 2020/223231, published Nov. 5, 2020, U.S. Patent Application No. 63/065,616, filed Aug. 14, 2020, and U.S. Patent Application No. 63/109,734, filed Nov. 4, 2020, which are incorporated herein by reference.

As used herein, “AAV9 capsid” refers to the AAV9 having the amino acid sequence of (a) GenBank accession: AAS99264, is incorporated by reference herein and the AAV vp1 capsid protein and/or (b) the amino acid sequence encoded by the nucleotide sequence of GenBank Accession: AY530579.1: (nt 1.2211). Some variation from this encoded sequence is encompassed by the present invention, which may include sequences having about 99% identity to the referenced amino acid sequence in GenBank accession: AAS99264 and U.S. Pat. No. 7,906,111 (also WO 2005/033321) (i.e., less than about 1% variation from the referenced sequence). Such AAV may include, e.g., natural isolates (e.g., hu31 or hu32), or variants of AAV9 having amino acid substitutions, deletions or additions, e.g., including but not limited to amino acid substitutions selected from alternate residues “recruited” from the corresponding position in any other AAV capsid aligned with the AAV9 capsid; e.g., such as described in U.S. Pat. Nos. 9,102,949, 8,927,514, US2015/349911, WO 2016/049230A1, U.S. Pat. Nos. 9,623,120, and 9,585,971. However, in other embodiments, other variants of AAV9, or AAV9 capsids having at least about 95% identity to the above-referenced sequences may be selected. See, e.g., US 2015/0079038. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

In certain embodiments, an AAVhu68 capsid is as described in WO 2018/160582, entitled “Novel Adeno-associated virus (AAV) Clade F Vector and Uses Therefor”, which is hereby incorporated by reference. In certain embodiments, AAVhu68 capsid proteins comprise: AAVhu68 vp1 proteins produced by expression from a nucleic acid sequence which encodes the predicted amino acid sequence of 1 to 736 of SEQ ID NO: 55, vp1 proteins produced from SEQ ID NO: 54 or vp1 proteins produced from a nucleic acid sequence at least 70% identical to SEQ ID NO: 54 which encodes the predicted amino acid sequence of 1 to 736 of SEQ ID NO: 55; AAVhu68 vp2 proteins produced by expression from a nucleic acid sequence which encodes the predicted amino acid sequence of at least about amino acids 138 to 736 of SEQ ID NO: 55, vp2 proteins produced from a sequence comprising at least nucleotides 412 to 2211 of SEQ ID NO: 54, or vp2 proteins produced from a nucleic acid sequence at least 70% identical to at least nucleotides 412 to 2211 of SEQ ID NO: 54 which encodes the predicted amino acid sequence of at least about amino acids 138 to 736 of SEQ ID NO: 55, and/or AAVhu68 vp3 proteins produced by expression from a nucleic acid sequence which encodes the predicted amino acid sequence of at least about amino acids 203 to 736 of SEQ ID NO: 55, vp3 proteins produced from a sequence comprising at least nucleotides 607 to 2211 of SEQ ID NO: 54, or vp3 proteins produced from a nucleic acid sequence at least 70% identical to at least nucleotides 607 to 2211 of SEQ ID NO: 54 which encodes the predicted amino acid sequence of at least about amino acids 203 to 736 of SEQ ID NO: 55.

The AAVhu68 vp1, vp2 and vp3 proteins are typically expressed as alternative splice variants encoded by the same nucleic acid sequence which encodes the full-length vp1 amino acid sequence of SEQ ID NO: 55 (amino acid 1 to 736). Optionally the vp1-encoding sequence is used alone to express the vp1, vp2, and vp3 proteins. Alternatively, this sequence may be co-expressed with one or more of a nucleic acid sequence which encodes the AAVhu68 vp3 amino acid sequence of SEQ ID NO: 55 (about aa 203 to 736) without the vp1-unique region (about aa 1 to about aa 137) and/or vp2-unique regions (about aa 1 to about aa 202), or a strand complementary thereto, the corresponding mRNA (about nt 607 to about nt 2211 of SEQ ID NO: 54), or a sequence at least 70% to at least 99% (e.g., at least 85%, at least 90%, at least 95%, at least 97%, at least 98% or at least 99%) identical to SEQ ID NO: 54 which encodes aa 203 to 736 of SEQ ID NO: 55. Additionally, or alternatively, the vp1-encoding and/or the vp2-encoding sequence may be co-expressed with the nucleic acid sequence which encodes the AAVhu68 vp2 amino acid sequence of SEQ ID NO: 55 (about aa 138 to 736) without the vp1-unique region (about aa 1 to about 137), or a strand complementary thereto, the corresponding mRNA (nt 412 to 2211 of SEQ ID NO: 54), or a sequence at least 70% to at least 99% (e.g., at least 85%, at least 90%, at least 95%, at least 97%, at least 98% or at least 99%) identical to nt 412 to 2211 of SEQ ID NO: 54 which encodes about aa 138 to 736 of SEQ ID NO: 55.

As described herein, a rAAVhu68 has a rAAVhu68 capsid produced in a production system expressing capsids from an AAVhu68 nucleic acid which encodes the vp1 amino acid sequence of SEQ ID NO: 55, and optionally additional nucleic acid sequences, e.g., encoding a vp3 protein free of the vp1 and/or vp2-unique regions. The rAAVhu68 resulting from production using a single nucleic acid sequence vp1 produces the heterogenous populations of vp1 proteins, vp2 proteins and vp3 proteins. More particularly, the AAVhu68 capsid contains subpopulations within the vp1 proteins, within the vp2 proteins and within the vp3 proteins which have modifications from the predicted amino acid residues in SEQ ID NO: 55. These subpopulations include, at a minimum, deamidated asparagine (N or Asn) residues. For example, asparagines in asparagine-glycine pairs are highly deamidated.

In one embodiment, the AAVhu68 vp1 nucleic acid sequence has the sequence of SEQ ID NO: 54, or a strand complementary thereto, e.g., the corresponding mRNA. In certain embodiments, the vp2 and/or vp3 proteins may be expressed additionally or alternatively from different nucleic acid sequences than the vp1, e.g., to alter the ratio of the vp proteins in a selected expression system. In certain embodiments, also provided is a nucleic acid sequence which encodes the AAVhu68 vp3 amino acid sequence of SEQ ID NO: 55 (about aa 203 to 736) without the vp1-unique region (about aa 1 to about aa 137) and/or vp2-unique regions (about aa 1 to about aa 202), or a strand complementary thereto, the corresponding mRNA (about nt 607 to about nt 2211 of SEQ ID NO: 54). In certain embodiments, also provided is a nucleic acid sequence which encodes the AAVhu68 vp2 amino acid sequence of SEQ ID NO: 55 (about aa 138 to 736) without the vp1-unique region (about aa 1 to about 137), or a strand complementary thereto, the corresponding mRNA (nt 412 to 2211 of SEQ ID NO: 54).

However, other nucleic acid sequences which encode the amino acid sequence of SEQ ID NO: 55 may be selected for use in producing rAAVhu68 capsids. In certain embodiments, the nucleic acid sequence has the nucleic acid sequence of SEQ ID NO: 54 or a sequence at least 70% to 99% identical, at least 75%, at least 80%, at least 85%, at least 90%, at least 95%, at least 97%, at least 99%, identical to SEQ ID NO: 54 which encodes SEQ ID NO: 55. In certain embodiments, the nucleic acid sequence has the nucleic acid sequence of SEQ ID NO: 54 or a sequence at least 70% to 99%, at least 75%, at least 80%, at least 85%, at least 90%, at least 95%, at least 97%, or at least 99% identical to about nt 412 to about nt 2211 of SEQ ID NO: 54 which encodes the vp2 capsid protein (about aa 138 to 736) of SEQ ID NO: 55. In certain embodiments, the nucleic acid sequence has the nucleic acid sequence of about nt 607 to about nt 2211 of SEQ ID NO: 54 or a sequence at least 70% to 99.%, at least 75%, at least 80%, at least 85%, at least 90%, at least 95%, at least 97%, or at least 99% identical to nt 412 to about nt 2211 of SEQ ID NO: 54 which encodes the vp3 capsid protein (about aa 203 to 736) of SEQ ID NO: 55.

It is within the skill in the art to design nucleic acid sequences encoding this AAVhu68 capsid, including DNA (genomic or cDNA), or RNA (e.g., mRNA). In certain embodiments, the nucleic acid sequence encoding the AAVhu68 vp1 capsid protein is provided in SEQ ID NO: 55. In certain embodiments, the AAVhu68 capsid is produced using a nucleic acid sequence of SEQ ID NO: 54 or a sequence at least 70%, at least 75%, at least 80%, at least 85%, at least 90%, at least 95%, at least 97%, or at least 99% which encodes the vp1 amino acid sequence of SEQ ID NO: 55 with a modification (e.g., deamidated amino acid) as described herein. In certain embodiments, the vp1 amino acid sequence is reproduced in SEQ ID NO: 55.

In certain embodiments, AAV capsids having reduced capsid deamidation may be selected. See, e.g., PCT/US19/19804 and PCT/US18/19861, both filed Feb. 27, 2019 and incorporated by reference in their entireties.

As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. SEQ ID NO: 55 provides the encoded amino acid sequence of the AAVhu68 vp1 protein. The term “heterogenous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, at least 97%, at least 99%, or up to about 100% deamidated at a referenced amino acid position, as compared to the predicted amino acid sequence at the reference amino acid position (e.g., at least 80% of the asparagines at amino acid 57 based on the numbering of SEQ ID NO: 55 [AAVhu68] may be deamidated based on the total vp1 proteins may be deamidated based on the total vp1, vp2 and vp3 proteins). Such percentages may be determined using 2D-gel, mass spectrometry techniques, or other suitable techniques.

Thus, an rAAV includes subpopulations within the rAAV capsid of vp1, vp2, and/or vp3 proteins with deamidated amino acids, including at a minimum, at least one subpopulation comprising at least one highly deamidated asparagine. In addition, other modifications may include isomerization, particularly at selected aspartic acid (D or Asp) residue positions. In still other embodiments, modifications may include an amidation at an Asp position.

In certain embodiments, an AAV capsid contains subpopulations of vp1, vp2 and vp3 having at least 4 to at least about 25 deamidated amino acid residue positions, of which at least 1 to 10% are deamidated as compared to the encoded amino acid sequence of the vp proteins. The majority of these may be N residues. However, Q residues may also be deamidated.

In certain embodiments, a rAAV has an AAV capsid having vp1, vp2 and vp3 proteins having subpopulations comprising combinations of two, three, four or more deamidated residues. Deamidation in the rAAV may be determined using 2D gel electrophoresis, and/or mass spectrometry, and/or protein modelling techniques. Online chromatography may be performed with an Acclaim PepMap column and a Thermo UltiMate 3000 RSLC system (Thermo Fisher Scientific) coupled to a Q Exactive HF with a NanoFlex source (Thermo Fisher Scientific). MS data is acquired using a data-dependent top-20 method for the Q Exactive HF, dynamically choosing the most abundant not-yet-sequenced precursor ions from the survey scans (200-2000 m/z). Sequencing is performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra may be set to 30,000 at m/z200 with a maximum ion injection time of 50 ms and a normalized collision energy of 30. The S-lens RF level may be set at 50, to give optimal transmission of the m/z region occupied by the peptides from the digest. Precursor ions may be excluded with single, unassigned, or six and higher charge states from fragmentation selection. BioPharma Finder 1.0 software (Thermo Fischer Scientific) may be used for analysis of the data acquired. For peptide mapping, searches are performed using a single-entry protein FASTA database with carbamidomethylation set as a fixed modification; and oxidation, deamidation, and phosphorylation set as variable modifications, a 10-ppm mass accuracy, a high protease specificity, and a confidence level of 0.8 for MS/MS spectra. Examples of suitable proteases may include, e.g., trypsin or chymotrypsin. Mass spectrometric identification of deamidated peptides is relatively straightforward, as deamidation adds to the mass of intact molecule +0.984 Da (the mass difference between —OH and —NH₂ groups). The percent deamidation of a particular peptide is determined by the mass area of the deamidated peptide divided by the sum of the area of the deamidated and native peptides. Considering the number of possible deamidation sites, isobaric species which are deamidated at different sites may co-migrate in a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent on the site of deamidation. It is understood by one of skill in the art that a number of variations on these illustrative methods can be used. For example, suitable mass spectrometers may include, e.g., a quadrupole time of flight mass spectrometer (QTOF), such as a Waters Xevo or Agilent 6530 or an orbitrap instrument, such as the Orbitrap Fusion or Orbitrap Velos (Thermo Fisher). Suitably liquid chromatography systems include, e.g., Acquity UPLC system from Waters or Agilent systems (1100 or 1200 series). Suitable data analysis software may include, e.g., MassLynx (Waters), Pinpoint and Pepfinder (Thermo Fischer Scientific), Mascot (Matrix Science), Peaks DB (Bioinformatics Solutions). Still other techniques may be described, e.g., in X. Jin et al, Hu Gene Therapy Methods, Vol. 28, No. 5, pp. 255-267, published online Jun. 16, 2017.

In addition to deamidations, other modifications may occur do not result in conversion of one amino acid to a different amino acid residue. Such modifications may include acetylated residues, isomerizations, phosphorylations, or oxidations.

Modulation of Deamidation: In certain embodiments, the AAV is modified to change the glycine in an asparagine-glycine pair, to reduce deamidation. In other embodiments, the asparagine is altered to a different amino acid, e.g., a glutamine which deamidates at a slower rate; or to an amino acid which lacks amide groups (e.g., glutamine and asparagine contain amide groups); and/or to an amino acid which lacks amine groups (e.g., lysine, arginine and histidine contain amine groups). As used herein, amino acids lacking amide or amine side groups refer to, e.g., glycine, alanine, valine, leucine, isoleucine, serine, threonine, cystine, phenylalanine, tyrosine, or tryptophan, and/or proline. Modifications such as described may be in one, two, or three of the asparagine-glycine pairs found in the encoded AAV amino acid sequence. In certain embodiments, such modifications are not made in all four of the asparagine-glycine pairs. Thus, a method for reducing deamidation of AAV and/or engineered AAV variants having lower deamidation rates. Additionally, or alternative one or more other amide amino acids may be changed to a non-amide amino acid to reduce deamidation of the AAV. In certain embodiments, a mutant AAV capsid as described herein contains a mutation in an asparagine-glycine pair, such that the glycine is changed to an alanine or a serine. A mutant AAV capsid may contain one, two or three mutants where the reference AAV natively contains four NG pairs. In certain embodiments, an AAV capsid may contain one, two, three or four such mutants where the reference AAV natively contains five NG pairs. In certain embodiments, a mutant AAV capsid contains only a single mutation in an NG pair. In certain embodiments, a mutant AAV capsid contains mutations in two different NG pairs. In certain embodiments, a mutant AAV capsid contains mutation is two different NG pairs which are located in structurally separate location in the AAV capsid. In certain embodiments, the mutation is not in the VP1-unique region. In certain embodiments, one of the mutations is in the VP1-unique region. Optionally, a mutant AAV capsid contains no modifications in the NG pairs, but contains mutations to minimize or eliminate deamidation in one or more asparagines, or a glutamine, located outside of an NG pair. In the AAVhu68 capsid protein, 4 residues (N57, N329, N452, N512) routinely display levels of deamidation >70% and it most cases >90% across various lots. Additional asparagine residues (N94, N253, N270, N304, N409, N477, and Q599) also display deamidation levels up to ˜20% across various lots. The deamidation levels were initially identified using a trypsin digest and verified with a chymotrypsin digestion.

The AAVhu68 capsid contains subpopulations within the vp1 proteins, within the vp2 proteins and within the vp3 proteins which have modifications from the predicted amino acid residues in SEQ ID NO: 55. These subpopulations include, at a minimum, certain deamidated asparagine (N or Asn) residues. For example, certain subpopulations comprise at least one, two, three or four highly deamidated asparagines (N) positions in asparagine-glycine pairs in SEQ ID NO: 55 and optionally further comprising other deamidated amino acids, wherein the deamidation results in an amino acid change and other optional modifications. The various combinations of these and other modifications are described herein.

In one aspect, provided herein is and AAV vector which comprises an AAV capsid and an expression cassette, wherein the expression cassette comprises a nucleic acid sequence encoding one more elements of a UBE3A-ATS gene editing system and regulatory elements that direct expression of the elements of the UBE3A-ATS gene editing in a host cell. The AAV vector also comprises AAV ITR sequences.

The ITRs are the genetic elements responsible for the replication and packaging of the genome during vector production and are the only viral cis elements required to generate rAAV. In one embodiment, the ITRs are from an AAV different than that supplying a capsid. In a preferred embodiment, the ITR sequences from AAV2, or the deleted version thereof (ΔITR), which may be used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, AAV vector genome comprises an AAV 5′ ITR, the nucleic acid sequences encoding the gene product(s) and any regulatory sequences, and an AAV 3′ ITR. However, other configurations of these elements may be suitable. In one embodiment, a self-complementary AAV is provided. A shortened version of the 5′ ITR, termed ΔITR, has been described in which the D-sequence and terminal resolution site (trs) are deleted. In certain embodiments, the vector genome includes a shortened AAV2 ITR of 130 base pairs, wherein the external “a” element is deleted. The shortened ITR is reverted back to the wild-type length of 145 base pairs during vector DNA amplification using the internal A element as a template. In other embodiments, the full-length AAV 5′ and 3′ ITRs are used.

In one embodiment, the regulatory sequences are selected such that the total rAAV vector genome is about 2.0 to about 5.5 kilobases in size. In one embodiment, the regulatory sequences are selected such that the total rAAV vector genome is about 2.9 to about 5.5 kilobases in size. In one embodiment, the regulatory sequences are selected such that the total rAAV vector genome is about 2.9 kb in size. In one embodiment, it is desirable that the rAAV vector genome approximate the size of the native AAV genome. Thus, in one embodiment, the regulatory sequences are selected such that the total rAAV vector genome is about 4.7 kb in size. In another embodiment, the total rAAV vector genome is less about 5.2 kb in size. The size of the vector genome may be manipulated based on the size of the regulatory sequences including the promoter, enhancer, intron, poly A, etc. See, Wu et al., Mol Ther, January 2010, 18(1):80-6, which is incorporated herein by reference.

In certain embodiments, provided herein is a rAAV useful as CNS-directed therapy for treatment of a subject having Angelman syndrome (AS), wherein the rAAV comprises an AAV capsid, and a vector genome packaged therein, said vector genome comprising: (a) an AAV 5′ inverted terminal repeat (ITR); (b) a sequence encoding components of a gene-editing system which is operably linked to regulatory elements which direct expression thereof in a host ell; (c) regulatory elements which direct expression; and (d) an AAV 3′ ITR. In one embodiment, the rAAV has a tropism for a cell of the CNS (e.g., an rAAV bearing an AAVhu68 capsid), and/or contains a neuron-specific expression control elements (e.g., a synapsin promoter). In one aspect, a construct is provided which is a vector (e.g., a plasmid) useful for generating viral vectors. In one embodiment, the AAV 5′ ITR is an AAV2 ITR and the AAV 3′ITR is an AAV2 ITR. In one embodiment, the rAAV comprises an AAV capsid as described herein. In one embodiment, the rAAV comprises an AAVhu68 capsid. In other embodiments, the rAAV comprises an AAV capsid provided that is not AAVhu68.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette as described herein flanked by AAV inverted terminal repeats (ITRs); and sufficient helper functions to permit packaging of the expression cassette into the AAV capsid protein. Also provided herein is the host cell which contains a nucleic acid sequence encoding an AAV capsid; a functional rep gene; a vector genome as described; and sufficient helper functions to permit packaging of the vector genome into the AAV capsid protein. In one embodiment, the host cell is a HEK 293 cell. These methods are described in more detail in WO2017160360 A2, which is incorporated by reference herein.

Other methods of producing rAAV available to one of skill in the art may be utilized. Suitable methods may include without limitation, baculovirus expression system or production via yeast. See, e.g., Robert M. Kotin, Large-scale recombinant adeno-associated virus production. Hum Mol Genet. 2011 Apr. 15; 20(R1): R2-R6. Published online 2011 Apr. 29. doi: 10.1093/hmg/ddr141; Aucoin M G et al., Production of adeno-associated viral vectors in insect cells using triple infection: optimization of baculovirus concentration ratios. Biotechnol Bioeng. 2006 Dec. 20; 95(6):1081-92; SAMI S. THAKUR, Production of Recombinant Adeno-associated viral vectors in yeast. Thesis presented to the Graduate School of the University of Florida, 2012; Kondratov O et al. Direct Head-to-Head Evaluation of Recombinant Adeno-associated Viral Vectors Manufactured in Human versus Insect Cells, Mol Ther. 2017 Aug. 10. pii: S1525-0016(17)30362-3. doi: 10.1016/j.ymthe.2017.08.003. [Epub ahead of print]; Mietzsch M et al, OneBac 2.0: Sf9 Cell Lines for Production of AAV1, AAV2, and AAV8 Vectors with Minimal Encapsidation of Foreign DNA. Hum Gene Ther Methods. 2017 February; 28(1):15-22. doi: 10.1089/hgtb.2016.164; Li L et al. Production and characterization of novel recombinant adeno-associated virus replicative-form genomes: a eukaryotic source of DNA for gene transfer. PLoS One. 2013 Aug. 1; 8(8):e69879. doi: 10.1371/journal.pone.0069879. Print 2013; Galibert L et al, Latest developments in the large-scale production of adeno-associated virus vectors in insect cells toward the treatment of neuromuscular diseases. J Invertebr Pathol. 2011 July; 107 Suppl:580-93. doi: 10.1016/j.jip.2011.05.008; and Kotin R M, Large-scale recombinant adeno-associated virus production. Hum Mol Genet. 2011 Apr. 15; 20(R1):R2-6. doi: 10.1093/hmg/ddr141. Epub 2011 Apr. 29.

A two-step affinity chromatography purification at high salt concentration followed by anion exchange resin chromatography are used to purify the vector drug product and to remove empty capsids. These methods are described in more detail in WO 2017/160360 entitled “Scalable Purification Method for AAV9”, which is incorporated by reference herein. In brief, the method for separating rAAV9 particles having packaged genomic sequences from genome-deficient AAV9 intermediates involves subjecting a suspension comprising recombinant AAV9 viral particles and AAV 9 capsid intermediates to fast performance liquid chromatography, wherein the AAV9 viral particles and AAV9 intermediates are bound to a strong anion exchange resin equilibrated at a pH of 10.2, and subjected to a salt gradient while monitoring eluate for ultraviolet absorbance at about 260 and about 280. Although less optimal for rAAV9, the pH may be in the range of about 10.0 to 10.4. In this method, the AAV9 full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2/9 serotype. Under these ionic conditions, a significant percentage of residual cellular DNA and proteins flow through the column, while AAV particles are efficiently captured.

Conventional methods for characterization or quantification of rAAV are available to one of skill in the art. To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles. Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Viral. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or Coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

Methods for determining the ratio among vp1, vp2, and vp3 of capsid protein are also available. See, e.g., Vamseedhar Rayaprolu et al, Comparative Analysis of Adeno-Associated Virus Capsid Stability and Dynamics, J Virol. 2013 December; 87(24): 13150-13160; Buller R M, Rose J A. 1978. Characterization of adenovirus-associated virus-induced polypeptides in KB cells. J. Virol. 25:331-338; and Rose J A, Maizel J V, Inman J K, Shatkin A J. 1971. Structural proteins of adenovirus-associated viruses. J. Virol. 8:766-770.

It should be understood that the compositions in the vectors described herein are intended to be applied to other compositions and methods described across the Specification.

### Compositions

Provided is an aqueous suspension suitable for administration to treat AS in a subject in need thereof, said suspension comprising an aqueous suspending liquid and vector comprising a nucleic acid sequence encoding one or more elements of a gene editing system operatively linked to regulatory elements therefor as described herein. In one embodiment, a therapeutically effective amount of said vector is included in the suspension.

### Nucleic Acids

In certain embodiments, the pharmaceutical composition comprises an expression cassette comprising the components of gene editing system and a non-viral delivery system. This may include, e.g., naked DNA, naked RNA, an inorganic particle, a lipid or lipid-like particle, a chitosan-based formulation and others known in the art and described for example by Ramamoorth and Narvekar, as cited above). In other embodiments, the pharmaceutical composition is a suspension comprising the expression cassette comprising the gene editing system in a viral vector system. In certain embodiments, the pharmaceutical composition comprises a non-replicating viral vector. Suitable viral vectors may include any suitable delivery vector, such as, e.g., a recombinant adenovirus, a recombinant lentivirus, a recombinant bocavirus, a recombinant adeno-associated virus (AAV), or another recombinant parvovirus. In certain embodiments, the viral vector is a recombinant AAV for delivery of a gene editing system for targeting UBE3A-ATS to a patient in need thereof.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

In one embodiment, the suspension further comprises a surfactant, preservative, excipients, and/or buffer dissolved in the aqueous suspending liquid. In one embodiment, the buffer is PBS. Various suitable solutions are known including those which include one or more of: buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration. A suitable surfactant, or combination of surfactants, may be selected from among Poloxamers, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. The pH may be in the range of 6.5 to 8.5, or 7 to 8.5, or 7.5 to 8. As the pH of the cerebrospinal fluid is about 7.28 to about 7.32, for intrathecal delivery, a pH within this range may be desired; whereas for intravenous delivery, a pH of 6.8 to about 7.2 may be desired. However, other pHs within the broadest ranges and these subranges may be selected for other routes of delivery.

Additionally provided is a pharmaceutical composition comprising a pharmaceutically acceptable carrier and a vector comprising a nucleic acid sequence encoding one or more components of a gene-editing system operatively linked to regulatory elements therefor as described herein. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered trangenes or rAAV vectors for delivery of one or more components of a CRISPR/Cas9 or other gene editing system may be formulated for delivery either encapsulated in a lipid particle, a liposome, a vesicle, a nanosphere, or a nanoparticle or the like. In one embodiment, a therapeutically effective amount of said vector is included in the pharmaceutical composition. Suitable carriers may be readily selected by one of skill in the art in view of the indication for which the vector is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention. Other conventional pharmaceutically acceptable carrier, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The phrase “pharmaceutically acceptable” refers to molecular entities and compositions that do not produce an allergic or similar untoward reaction when administered to a host.

As used herein, the term “dosage” or “amount” can refer to the total dosage or amount delivered to the subject in the course of treatment, or the dosage or amount delivered in a single unit (or multiple unit or split dosage) administration.

The aqueous suspension or pharmaceutical compositions described herein are designed for delivery to subjects in need thereof by any suitable route or a combination of different routes. In one embodiment, the pharmaceutical composition comprises an expression cassette or vector described herein in a formulation buffer suitable for delivery via intracerebroventricular (ICV), intrathecal (IT), intracisternal, or intravenous (IV) routes of administration. Alternatively, other routes of administration may be selected (e.g., oral, inhalation, intranasal, intratracheal, intraarterial, intraocular, intramuscular, and other parenteral routes).

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular, suboccipital/intracisternal, and/or C1-2 puncture. For example, material may be introduced for diffusion throughout the subarachnoid space by means of lumbar puncture. In another example, injection may be into the cisterna magna. Intracisternal delivery may increase vector diffusion and/or reduce toxicity and inflammation caused by the administration. See, e.g., Christian Hinderer et al, Widespread gene transfer in the central nervous system of cynomolgus macaques following delivery of AAV9 into the cisterna magna, Mol Ther Methods Clin Dev. 2014; 1: 14051. Published online 2014 Dec. 10. doi: 10.1038/mtm.2014.51.

As used herein, the terms “intracisternal delivery” or “intracisternal administration” refer to a route of administration for drugs directly into the cerebrospinal fluid of the brain ventricles or within the cisterna magna cerebellomedularis, more specifically via a suboccipital puncture or by direct injection into the cisterna magna or via permanently positioned tube.

In one aspect, provided herein is a pharmaceutical composition comprising a vector as described herein in a formulation buffer. In certain embodiments, the replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10″ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

In one embodiment, provided is a pharmaceutical composition comprising a rAAV as described herein in a formulation buffer. In one embodiment, the rAAV is formulated at about 1×10⁹ genome copies (GC)/mL to about 1×10¹⁴ GC/mL. In a further embodiment, the rAAV is formulated at about 3×10⁹ GC/mL to about 3×10¹³ GC/mL. In yet a further embodiment, the rAAV is formulated at about 1×10⁹ GC/mL to about 1×10¹³ GC/mL. In one embodiment, the rAAV is formulated at least about 1×10¹¹ GC/mL.

Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

In the case of AAV viral vectors, quantification of the genome copies (“GC”) may be used as the measure of the dose contained in the aqueous suspension or pharmaceutical compositions. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR or quantitative PCR using primer/probe sets targeting specific region of the viral genome (usually poly A signal). The replication-defective virus compositions can be formulated in dosage units to contain an amount of replication-defective virus that is in the range of about 1.0×10⁹ GC to about 1.0×10¹⁵ GC, and preferably 1.0×10¹² GC to 1.0×10¹⁴ GC for a human patient. Preferably, the concentration of replication-defective virus in the formulation is about 1.0×10⁹ GC, about 5.0×10⁹ GC, about 1.0×10¹⁰ GC, about 5.0×10¹⁰ GC, about 1.0×10¹¹ GC, about 5.0×10¹¹ GC, about 1.0×10¹² GC, about 5.0×10¹² GC, about 1.0×10¹³ GC, about 5.0×10¹³ GC, about 1.0×10¹⁴ GC, about 5.0×10¹⁴ GC, or about 1.0×10¹⁵ GC. Alternative or additional method for performing AAV GC number titration is via oqPCR or digital droplet PCR (ddPCR) as described in, e.g., M. Lock et al, Hum Gene Ther Methods. 2014 April; 25(2):115-25. doi: 10.1089/hgtb.2013.131. Epub 2014 Feb. 14, which is incorporated herein by reference.

It should be understood that the compositions in the pharmaceutical compositions described herein are intended to be applied to other compositions, regimens, aspects, embodiments, and methods described across the Specification

### Methods

In certain embodiments, an expression cassette, nucleic acid, or a viral or non-viral vector is used in preparing a medicament. In certain embodiments, uses of the same for treatment of Angelman syndrome in a subject in need thereof are provided.

As used herein, the term “treatment” or “treating” is defined encompassing administering to a subject one or more compounds or compositions described herein for the purposes of amelioration of one or more symptoms of UBE3A deficiency or Angelman syndrome (AS). “Treatment” can thus include one or more of reducing onset or progression of AS, preventing disease, reducing the severity of the disease symptoms, retarding their progression, removing the disease symptoms, delaying progression of disease, or increasing efficacy of therapy in a given subject.

A goal of therapies described herein is to enhance UBE3A expression to achieve a desired result, i.e., treatment of Angelman syndrome (AS) or one or more symptoms thereof. Such symptoms may include but are not limited to one of more of the following: intellectual disability, speech impairment, ataxia, epilepsy, seizure disorder, microcephaly, psychomotor delay, and muscular hypotonia with hyperreflexia (See e.g., K. Buiting, et al., Nature reviews. Neurology, (2016), which is incorporated herein by reference). As described herein, a desired result may include reducing or eliminating neurophysical complications including delayed development, intellectual disability, severe speech impairment, and problems with movement and balance.

A “therapeutically effective amount” of a composition provided herein is delivered to a subject to achieve a desired result or to reach a therapeutic goal. In one embodiment, a therapeutic goal for treating AS is to restore UBE3A expression in a neuron, or in a population of neurons, to the functional level in a patient that is in the normal range or to the non-AS level. In another embodiment, therapeutic goal for treatment of AS is to increase the UBE3A expression to at least about 99%, about 95%, about 90%, about 85%, about 80%, about 75%, about 70%, about 65%, about 60%, about 55%, about 50%, about 45%, about 40%, about 45%, about 40%, about 35%, about 30% about 25%, about 20%, about 15%, about 10%, about 5%, about 2%, about 1% of the normal or non-AS level, or as compared to levels of UBE3A expression before treatment. Patients rescued by delivering UBE3A function to less than 100% activity levels may optionally be subject to further treatment. In another embodiment, therapeutic goals for treatment of AS are to increase the UBE3A expression in a percentage of target neurons, including about 60%, about 55%, about 50%, about 45%, about 40%, about 45%, about 40%, about 35%, about 30% about 25%, about 20%, about 15%, about 10%, about 5%, about 2%, or about 1% of neurons in a selected population.

In certain embodiments, provided herein is a method of treating AS by administering to a subject in need thereof an expression cassette, vector, or rAAV that provides one or more elements of a gene editing system editing, wherein editing of UBE3A-ATS results in enhanced expression of UBE3A from a paternal allele in a neuron. In certain embodiments, the method includes delivering a nucleic acid sequence which expresses a nuclease which binds to a sequence in UBE3A-ATS downstream of the UBE3A 3′UTR. Without wishing to be bound by theory, editing of the UBE3A-ATS coding sequence unsilences UBE3A expression on a paternal allele of a patient having a deficiency in UBE3A expression from a maternal allele and provided for expression of the UBE3A gene product from the paternal allele. In certain embodiments, the gene editing system introduces a mutation or modification that is an indel, deletion, insertion, inversion, or other disruption that interferes with transcription of the UBE3A-ATS coding sequence. In certain embodiments, the method includes introducing a mutation in the human UBE3A-ATS in the region spanning the UBE3A 3′UTR and SNORD109B. In certain embodiments, the mutation is introduced in a target sequence located at chr15: 25,278,409-25,333,728 (hg38 genome assembly) and/or in a sequence of UBE3A-ATS complementary to the region between the UBE3A 3′UTR and SNORD109B ORF on chromosome 15.

The gene therapy described herein, whether viral or non-viral, may be used in conjunction with other treatments (secondary therapy), i.e., the standard of care for the subject's (patient's) diagnosis and condition. As used herein, the term “secondary therapy” refers to the therapy that could be combined with the gene therapy described herein for the treatment of AS. In some embodiments, the gene therapy described herein is administered in combination with one or more secondary therapies for the treatment of AS, such as administering an anticonvulsant or dietary restriction (e.g., ketogenic and low glycemic). The secondary therapy may be any therapy which helps prevent, arrest or ameliorate these symptoms of AS. The secondary therapy can be administered before, concurrent with, or after administration of the compositions described above. Subjects may be permitted to continue their standard of care treatment(s) prior to and concurrently with the gene therapy treatment at the discretion of their caring physician. In the alternative, the physician may prefer to stop standard of care therapies prior to administering the gene therapy treatment and, optionally, resume standard of care treatments as a co-therapy after administration of the gene therapy. In another embodiment, the gene therapy described herein may be combined with genotypic analysis or genetic screening, which is routine in the art and may include the use of PCR to identify one or more mutations in the nucleic acid sequence of the UBE3A gene. As discussed above, subjects showing symptoms of AS early in life (e.g. 1-3 months) as well as subjects diagnosed with AS later in life are the intended recipients of the compositions and methods described herein.

By “administering” or “route of administration” is delivery of composition described herein, with or without a pharmaceutical carrier or excipient, of the subject. Routes of administration may be combined, if desired. In some embodiments, the administration is repeated periodically. Sequential administration may imply a time gap of multi-administration from intervals of days, weeks, months or years. In one embodiment, the compositions described herein are administered to a subject in need for one or more times. In one embodiment, the administrations are days, weeks, months or years apart. In one embodiment, one, two, three or more re-administrations are permitted. Such re-administration may be with the same type of vector, or a different vector. In a further embodiment, the vectors described herein may be used alone, or in combination with the standard of care for the patient's diagnosis and condition. The nucleic acid molecules and/or vectors described herein may be delivered in a single composition or multiple compositions. Optionally, two or more different AAV may be delivered, or multiple viruses [see, e.g., WO 2011/126808 and WO 2013/049493].

In one embodiment, the expression cassette, vector, or other composition described herein for gene therapy is delivered as a single dose per patient. In one embodiment, the subject is delivered a therapeutically effective amount of a composition described herein. As used herein, a “therapeutically effective amount” refers to the amount of the expression cassette or vector, or a combination thereof.

In one embodiment, the expression cassette is in a vector genome delivered in an amount of about 1×10⁹ GC per gram of brain mass to about 1×10¹³ genome copies (GC) per gram (g) of brain mass, including all integers or fractional amounts within the range and the endpoints. In another embodiment, the dosage is 1×10¹⁰ GC per gram of brain mass to about 1×10¹³ GC per gram of brain mass. In specific embodiments, the dose of the vector administered to a patient is at least about 1.0×10⁹ GC/g, about 1.5×10⁹ GC/g, about 2.0×10⁹ GC/g, about 2.5×10⁹ GC/g, about 3.0×10⁹ GC/g, about 3.5×10⁹ GC/g, about 4.0×10⁹ GC/g, about 4.5×10⁹ GC/g, about 5.0×10⁹ GC/g, about 5.5×10⁹ GC/g, about 6.0×10⁹ GC/g, about 6.5×10⁹ GC/g, about 7.0×10⁹ GC/g, about 7.5×10⁹ GC/g, about 8.0×10⁹ GC/g, about 8.5×10⁹ GC/g, about 9.0×10⁹ GC/g, about 9.5×10⁹ GC/g, about 1.0×10¹⁰ GC/g, about 1.5×10¹⁰ GC/g, about 2.0×10¹⁰ GC/g, about 2.5×10¹⁰ GC/g, about 3.0×10¹⁰ GC/g, about 3.5×10¹⁰ GC/g, about 4.0×10¹⁰ GC/g, about 4.5×10¹⁰ GC/g, about 5.0×10¹⁰ GC/g, about 5.5×10¹⁰ GC/g, about 6.0×10¹⁰ GC/g, about 6.5×10¹⁰ GC/g, about 7.0×10¹⁰ GC/g, about 7.5×10¹⁰ GC/g, about 8.0×10¹⁰ GC/g, about 8.5×10¹⁰ GC/g, about 9.0×10¹⁰ GC/g, about 9.5×10¹⁰ GC/g, about 1.0×10¹¹ GC/g, about 1.5×10¹¹ GC/g, about 2.0×10¹¹ GC/g, about 2.5×10¹¹ GC/g, about 3.0×10¹¹ GC/g, about 3.5×10¹¹ GC/g, about 4.0×10¹¹ GC/g, about 4.5×10¹¹ GC/g, about 5.0×10¹¹ GC/g, about 5.5×10¹¹ GC/g, about 6.0×10¹¹ GC/g, about 6.5×10¹¹ GC/g, about 7.0×10¹¹ GC/g, about 7.5×10¹¹ GC/g, about 8.0×10¹¹ GC/g, about 8.5×10¹¹ GC/g, about 9.0×10¹¹ GC/g, about 9.5×10¹¹ GC/g, about 1.0×10¹² GC/g, about 1.5×10¹² GC/g, about 2.0×10¹² GC/g, about 2.5×10¹² GC/g, about 3.0×10¹² GC/g, about 3.5×10¹² GC/g, about 4.0×10¹² GC/g, about 4.5×10¹² GC/g, about 5.0×10¹² GC/g, about 5.5×10¹² GC/g, about 6.0×10¹² GC/g, about 6.5×10¹² GC/g, about 7.0×10¹² GC/g, about 7.5×10¹² GC/g, about 8.0×10¹² GC/g, about 8.5×10¹² GC/g, about 9.0×10¹² GC/g, about 9.5×10¹² GC/g, about 1.0×10¹³ GC/g, about 1.5×10¹³ GC/g, about 2.0×10¹³ GC/g, about 2.5×10¹³ GC/g, about 3.0×10¹³ GC/g, about 3.5×10¹³ GC/g, about 4.0×10¹³ GC/g, about 4.5×10¹³ GC/g, about 5.0×10¹³ GC/g, about 5.5×10¹³ GC/g, about 6.0×10¹³ GC/g, about 6.5×10¹³ GC/g, about 7.0×10¹³ GC/g, about 7.5×10¹³ GC/g, about 8.0×10¹³ GC/g, about 8.5×10¹³ GC/g, about 9.0×10¹³ GC/g, about 9.5×10¹³ GC/g, or about 1.0×10¹⁴ GC/g brain mass.

In certain embodiments, treatment of a subject having AS with a composition described herein to introduce mutation (e.g., indel) in UBE3A-ATS may not require readministration. Alternatively, a second or subsequent additional treatment that includes a composition comprising a gene editing system provided herein may be pursued. Such subsequent treatment may utilize vectors having different capsids than were utilized for the initial treatment. Still other combinations of AAV capsids may be selected by one skilled in the art.

It is desirable that the lowest effective concentration of virus or other delivery vehicle be utilized in order to reduce the risk of undesirable effects, such as toxicity. Still other dosages in these ranges may be selected by the attending physician, taking into account the physical state of the subject, preferably human, being treated, the age of the subject, and the degree to which the disorder, if progressive, has developed.

Generally, the methods include administering to a mammalian subject in need thereof, a pharmaceutically effective amount of a composition comprising a recombinant adeno-associated virus (AAV) carrying a nucleic acid sequence encoding one or more elements of a UBE3A-ATS gene editing system under the control of regulatory sequences, and a pharmaceutically acceptable carrier. In one embodiment, such a method is designed for treating, retarding or halting progression of AS in a mammalian subject.

In one embodiment, provided is a method of treating AS by administering to a subject in need the vector, the rAAV, the aqueous suspension, or the pharmaceutical composition as described in the present specification. In one embodiment, a rAAV is delivered about 1×10¹⁰ to about 1×10¹⁵ genome copies (GC)/kg body weight. In certain embodiments, the subject is human. In one embodiment, the rAAV is administered more than one time. In a further embodiment, the rAAV is administered days, weeks, months or years apart.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1— Materials and Methods

### Plasmid Construction

We replaced the TBG promoter in the previously described pAAV.U6.sgRNA.TBG.SaCas9 vector (Yang Y, et al. Nat Biotechnol. 2016; 34(3):334-8) with the human synapsin I promoter (Thiel G, et al. Proc Natl Acad Sci USA. 1991; 88(8):3431-5) to obtain the pAAV.U6.sgRNA.hSyn.SaCas9 vector. We selected 12 20-bp target sequences preceding a 5′NNGRTT PAM sequence for Ube3α-ATS gene editing and subcloned them into the sgRNA cloning site (see Table 1 for details). The target sequences sampled a 12-kbp region downstream of the Ube3a 3′UTR (chr7:59,341,000-59,353,000, GRCm38/mm10 genome assembly). The nontargeted sequence consisted of a scrambled 20-bp sequence (5′-GAGACGGTCTTCGACGTCTC-3′, SED ID NO: 56). The nuclease-deficient dCas9 mutant was generated by point mutagenesis (D10A and H840A). We screened the target sequences in vitro with Neuro2a cells (ATCC, Manassas, Va.) using plasmid transfections with TransIT-LT1 (MirusBio, Madison, Wis.) per the manufacturer's instructions. After isolating gDNA (QiaAmp DNA Mini kit, Qiagen, Waltham, Mass.), we amplified the respective target regions by PCR and quantified indel frequencies by Amplicon-Seq. We selected the following target sequence for in vivo studies (sgRNA #7):

### 5′-TTGCCCAACCTCTCAAACGT-3′ (SEQ ID NO: 35).

### Sequencing Analysis

Amplicon-Seq: We amplified the region of interest with a forward and reverse primer pair (Table 1) using Q5 High-Fidelity DNA Polymerase (New England Biolabs, Ipswich, Mass.) according to the manufacturer's instructions. NGS libraries were produced with the 191-bp product and sequenced using an Illumina MiSeq Reagent V2 kit (150-bp pair-end, Illumina, San Diego, Calif.) as previously described (Wang L, et al. Nat Biotechnol. 2018; 36(8):717-25). We mapped read pairs to the mouse-reference genome using NovoAlign (Novocraft, Selangor, Malaysia); we retained reads mapping to the target region for further analysis. We determined the number of reads containing indels in a 40-bp target area (chr7:59346110-59346150, mouse genome assembly GRCm38.p6) using a custom script and reported that number as a percentage of the total number of reads mapping to the target region (indel %).

TABLE 1
In vivo off-target analysis for gene editing with selected sgRNA for
aged mice Ube3am+/p− (maternal Ube3a-ko) mice were injected
with an AAV vector encoding CRISPR/Cas9 at birth (day 0), and the cerebral
cortices were harvested four months later. We conducted ITR-seq to detect
off-target gene editing (5 mice per group). Genes or predicted genes
at the off-target location were identified using the USCD genome browser
(mm 10 genome assembly). Both introns and exons were queried.

| Group | On-target | On- target location(s) | Off-target | Off- target location(s) |
|---|---|---|---|---|
| Non-targeted | 0% | — | 100% | chr2: 98666626 |
| (NT) Cas9 | 0 reads | | 14 reads | (exon 5 of predicted |
| vector | | | | gene Gm10800) |
| | | | | chr5: 121718135 (intron |
| | | | | 1 of Atxn2) |
| Targeted | 99.5% | chr7: 59346117 | 0.5% | chr2: 98667059 |
| Cas9 vector | 7427 reads | (Ube3a-ATS) | 40 reads | (intron 3 of predicted |
| | | | | gene Gm10800) |
| | | | | chr5: 6351200 (no |
| | | | | known gene) |

AMP-Seq: We performed anchored multiplexed PCR sequencing (AMP-Seq) analysis as follows: A sample of 500 ng of genomic DNA was sheared using a Covaris ME220 instrument, and the DNA was end-repaired, A-tailed, and ligated to adapters as previously reported (Wang L, et al. Nat Biotechnol. 2018; 36(8):717-25; Zheng Z, et al. Nat Med. 2014; 20(12):1479-84). We amplified the region of interest using GSP1 and GSP2 primers in first and second nested PCR reactions, respectively: 95° C.-5 min, 15 cycles of [95° C.-30 s, 70° C. (−1° C./cycle)-2 min, 72° C.-30 s], 10 cycles of [95° C.-30 s, 55° C.-1 min, 72° C.-30 s], 72° C.-5 min.

### Primers:

ANG3MGSP1P
(SEQ ID NO: 38)
5′-AGCTGCCCAAGCACTTATAGACATGAC-3′,
ANG3MGSP2P
(SEQ ID NO: 39)
5′-CCTCTCTATGGGCAGTCGGTGAAAAATCT
CCCTAGAATTCAGGGCCGAGG-3′,
ANG3MGSP1N
(SEQ ID NO: 40)
5′-ATTTTTACGTTTGTTCCCTCCATCTTCC-3′,
ANG3MGSP2N
(SEQ ID NO: 41)
5′-CCTCTCTATGGGCAGTCGGTGAACCTAC
ACATTTGGTTGAAACAGGGAAGGC-3′

Libraries were constructed and sequenced on MiSeq (Illumina) as previously describe. We quantified reads containing insertions, deletions, translocations, and insertions corresponding to the AAV vector using our own custom script (Wang L, et al. Nat Biotechnol. 2018; 36(8):717-25).

ITR-Seq: Off-target editing mediated by the sgRNA+SaCas9 complex was determined by inverted terminal repeat sequencing (ITR-Seq) (Breton C, et al. BMC Genomics. 2020; 21(1):239). Briefly, the DNA was sheared, end-repaired, A-tailed, and ligated to adapters containing unique molecular barcodes. The DNA was then amplified by two rounds of PCR using an AAV-ITR-specific primer and adapter-specific primers, resulting in NGS-compatible libraries, which were subsequently sequenced on MiSeq (Illumina). We used a custom script to identify the genomic locations (including intronic and exonic locations) of AAV integration sites that resulted from double-strand breaks (Breton C, et al. BMC Genomics. 2020; 21(1):239).

### AAV Vector Production

All AAVhu68 (Hinderer C, et al. Hum Gene Ther. 2018; 29(3):285-98) and AAV9-PHP.B (Deverman B E, et al. Nat Biotechnol. 2016; 34(2):204-9) vectors were produced as previously described (Lock M, et al. Hum Gene Ther. 2010; 21(10):1259-71). In brief, HEK293 cells were triple transfected and the culture supernatant was harvested, concentrated, and purified with an iodixanol gradient. The purified vectors were titrated with droplet digital PCR using primers targeting the bovine growth hormone polyadenylation sequence as previously described (Lock M, et al. Hum Gene Ther Methods. 2014; 25(2):115-25).

### Animals

We purchased C56BL/6J (stock no. 000664), B6.129S7-Ube3α^{tm1alb}/J (016590), and B6.129S7-Ube3α^{tm2alb}/J (017765) mice from the Jackson Laboratory and maintained the animals at the University of Pennsylvania. Experimental cohorts were generated by crossing female C56BL/6J mice with male B6.129S7-Ube3α^{tm2alb}/J mice or male C56BL/6J mice with female B6.129S7-Ube3α^{tm1alb}/J mice. For gene-editing experiments, we randomized the litters and injected 1 μl of AAVhu68 vector in anesthetized neonatal mice in each lateral ventricle for a total of 1×10¹¹ gc. Anesthetized juvenile mice (postnatal day 14, 21, or 28) were intravenously (IV) retro-orbitally injected in the right eye with 50 μl AAV9-PHP.B vector for a total of 1×10¹² gc. The AAV vector was diluted in Dulbecco's phosphate-buffered saline (DPBS) to achieve the appropriate dose. The animals were housed in standard caging with two to five animals per cage. Cages, water bottles, and bedding substrates were autoclaved in the barrier facility, and cages were changed once per week. An automatically controlled 12-h light/dark cycle was maintained, with each dark period beginning at 6:00 p.m. Autoclaved laboratory rodent food and chlorinated water were provided ad libitum. At weaning age, ear tags with a unique four-digit number were applied and mice were randomly distributed in cages of approximately equal group sizes. A tissue sample was processed for genotyping. All researchers involved in observing the mice, processing samples, and analyzing data were blinded to the genotype and treatment group. Data was grouped after initial analysis to apply statistical tests.

### Vector Biodistribution

We extracted tissue DNA with a QIAamp DNA Mini Kit (Qiagen, Germantown, Md.) and quantified vector genomes by real-time PCR using TaqMan reagents (Thermo Fisher Scientific, Waltham, Mass.), primers, and probes targeting the bovine growth hormone sequence of the vector.

### Western Blotting

We lysed frozen cerebral cortices in RIPA buffer and separated 45 μg of total protein using SDS-PAGE. Following transfer, we blocked PVDF membranes with 1% BSA in TPBS (PBS+0.1% Tween) and incubated overnight with one of the following antibodies: anti-Ube3a mAb (611416, BD Biosciences, San Jose, Calif.), anti-GFP pAb (GFP-1010, Ayes Labs, Davis, Calif.), or anti-beta Actin mAb (MA5-15739, Invitrogen, Thermo Fisher, Waltham, Mass.). After incubation with secondary antibodies conjugated to HRP, we visualized the Western blot with a Clarity Western ECL Substrate kit (170-5060, Bio-Rad, Hercules, Calif.) on a Bio-Rad ChemiDoc imaging system. Each individual Western blotting experiment was carried out independently at least three times.

### Histology

Mice were anesthetized and terminally perfused with DPBS, and the whole brain was promptly collected. One half of a sagittally sectioned brain was immersion-fixed in 10% neutral-buffered formalin for approximately 24 h, washed briefly in PBS, and equilibrated in 70% ethanol before being embedded in paraffin and cut into 10-μm-thick sections. We snap-froze the other half of the brain on dry ice for biochemical analysis. We performed immunofluorescence for YFP and immunohistochemistry for UBE3A on formalin-fixed, paraffin-embedded brain samples. For immunofluorescence staining, the sections were deparaffinized, boiled for 6 min in 10 mM citrate buffer (pH 6.0) for antigen retrieval, blocked with 1% donkey serum in PBS+0.2% Triton for 15 min, and then incubated with anti-GFP antibodies (A-11122, Invitrogen, Thermo Fisher Scientific, Waltham, Mass.) and anti-NeuN antibodies (ABN90, Sigma-Aldrich, St. Louis, Mich.) at 1:500 dilution for 1 h. After being washed, the samples were incubated for 45 min with fluorescence-labeled secondary antibodies (anti-rabbit IgG-Alexa488 and anti-guinea pig IgG-Cy5 conjugates at a 1:200 dilution [Jackson ImmunoResearch, West Grove, Pa.]). For immunohistochemical staining, the sections were deparaffinized, boiled for 6 min in 10 mM citrate buffer (pH 6.0) for antigen retrieval, and sequentially treated with 2% H₂O₂ (15 min), avidin and biotin blocking reagents (15 min each; Vector Laboratories, Burlingame, Calif.), and blocking buffer (1% donkey serum in PBS with 0.2% Triton for 10 min). We then incubated the sections with anti-UBE3A mouse antibodies (611416, BD Biosciences, San Jose, Calif.) at 1:500 for 1 h and then with a biotinylated secondary antibody (Jackson ImmunoResearch, West Grove, Pa.) diluted in blocking buffer for 45 min. We used a Vectastain Elite ABC Kit (Vector Laboratories) according to the manufacturer's instructions, with 3,3′-diaminobenzidine as the substrate to stain bound antibodies as a brown precipitate. We captured images with a Nikon Eclipse Ti-E microscope, and scanned whole-brain sections with an Aperio Versa slide scanner.

### Gene-Expression Analysis

RNA was extracted from flash-frozen cerebral cortices with RNAeasy and DNAseI kits (Qiagen, Germantown, Md.), followed by cDNA synthesis (Maxima First Strand cDNA Synthesis Kit, Thermo Fisher, Waltham, Mass.). We performed SYBR green qPCR (Thermo Fisher) in triplicate for each sample with previously published primers (Meng L, et al. Nature. 2015; 518(7539):409-12) or primers listed in Table 1 according to the manufacturer's instructions for an ABI7500 thermocycler (Thermo Fisher).

### Behavioral Assessment

The behavioral phenotype of maternal Ube3α-KO mice has been well characterized (12-16) and can be improved by genetically restoring maternal Ube3a expression (Sonzogni M, et al. Molecular autism. 2018; 9:47). The mice were group-housed after weaning, mixed by genotype and treatment. We determined the weight of each animal a few days before starting the behavioral analysis. Prior to each test, the mice were acclimatized to the testing room in their home cage for 30 min. All behavioral experiments were performed during the afternoon light period of the light/dark cycle. We used both male and female mice aged 8-10 weeks for the experiments. After testing, the mice were promptly returned to the holding room. The housing cages were composed of clear polycarbonate plastic (7.75×12×5 inches). Data presented is based on accumulating results from three independent experimental cohorts. The same set of breeders was used to generate those experimental cohorts.

Accelerating rotarod: We tested motor function using an accelerating rotarod (4-40 rpm in 5 min; model 7650, Ugo Basile Biological Research Apparatus, Varese, Italy). The mice were subjected to three trials per day with a 15-min intertrial interval for three consecutive days (same time each day). For each day, we calculated the average time spent by the mouse on the rotarod until falling off (latency in seconds). If a mouse achieved three consecutive wrapping/passive rotations on the rotarod, the time after the third rotation was recorded as the latency, and the mouse was removed.

Open-field activity test: To test locomotor activity, we individually placed mice in a new housing cage with a minimal amount of bedding covering the bottom. The cage was placed in an array of infrared cross beams (Med Associates, Inc., Fairfax, Vt.). We allowed the mice to freely explore for 30 min, with the number of beam breaks automatically recorded as a measure of activity. The numbers of beam breaks were summed in bins with a duration of 5 min for analysis.

Marble burying test: Housing cages were filled with 5 cm of bedding material (Alpha-Dri, Lab Supply, Fort Worth, Tex.). On top of the bedding material, we arranged 12 blue glass marbles arranged in an equidistant 3×4 grid. We gave the animals access to the marbles for 30 min. After the test, the mice were removed from the cage, and the marbles that were more than 50% covered by bedding were scored as buried. The outcome measured for this test was the number of buried marbles.

Nest-building test: To measure nest-building ability, mice were singly housed in a new cage and provided with a pre-weighed square nestlet (2×2×0.25 inches). After 24 h, the mice were returned to their original home cage, and the quality of the nest was scored on a scale of 1 to 5, as previously described (Deacon R M. Nature protocols. 2006; 1(3):1117-9). The remaining untorn nestlet was weighed to calculate the fraction of remaining nestlet relative to the original weight.

### Statistics

We used Prism 8 software (GraphPad, San Diego, Calif.) for statistical analysis. We compared multiple groups by applying one- or two-way ANOVA F-tests followed by post-hoc Tukey's or Sidak's pairwise comparison with an alpha value of 0.05. Group averages are presented as the mean+/−SEM. If less than 10 data points per group are present, individual data points are shown in the graph as well. All data points obtained were used for statistical analysis.

### Example 2— Restored Expression of Paternal UBE3A Following Editing of UBE3A Antisense Transcript

Manipulation of the genomic sequence by gene editing is a powerful tool to correct genetic mutations but has largely been inaccessible for the in vivo use in post-mitotic cells such as neurons. However, gene editing can also be used as a cell-type independent tool to disrupt the genetic code by base pair deletion and insertion, termed indel formation. Here we show that indel formation within the Ube3α-ATS sequence downstream of the Ube3a gene locus is able to prevent extension of murine Ube3α-ATS across the Ube3 gene locus, to cause paternal Ube3a expression and to improve the Angelman phenotype in maternal Ube3a-deficient mice.

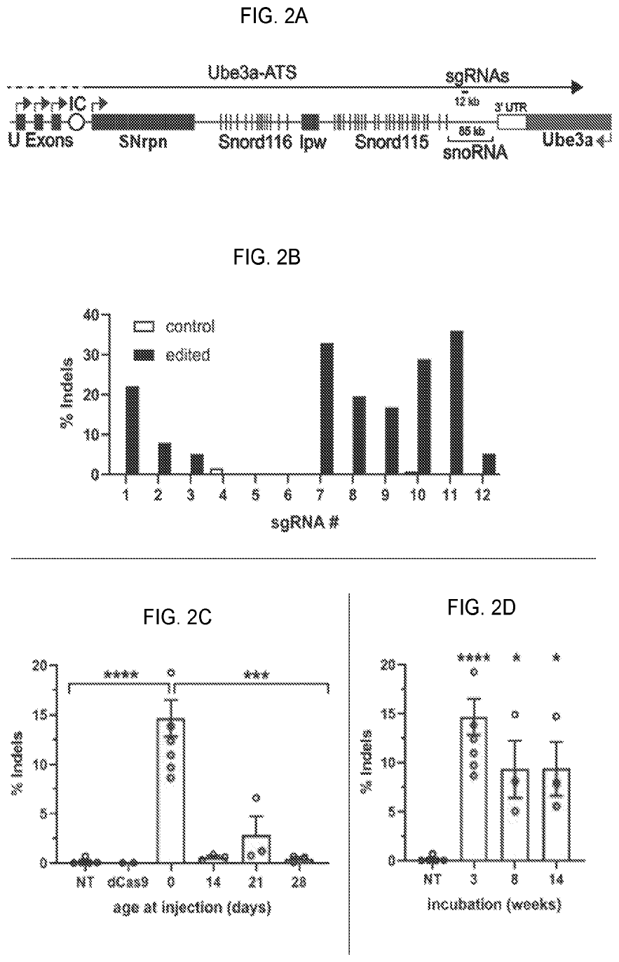

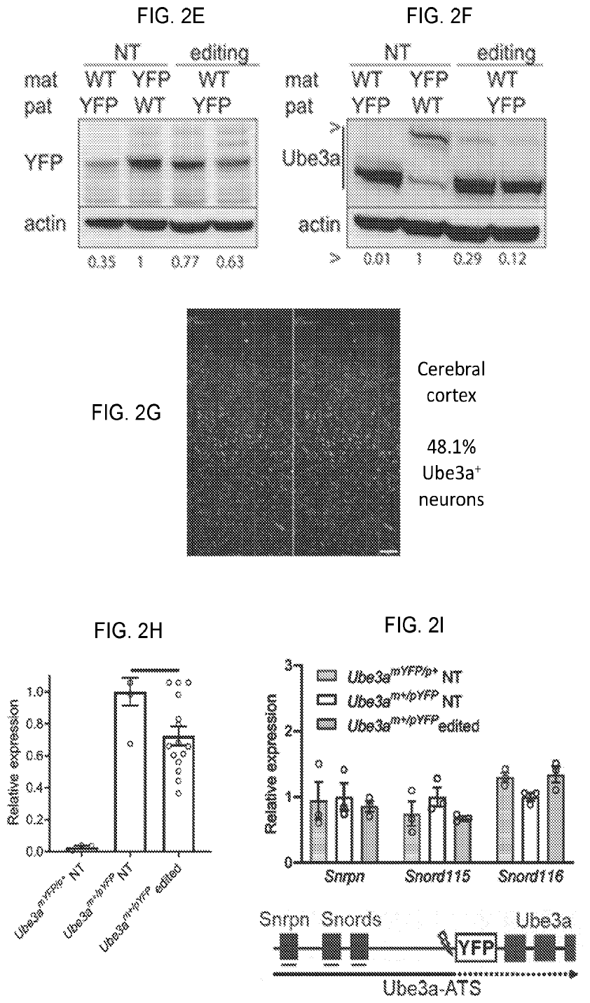

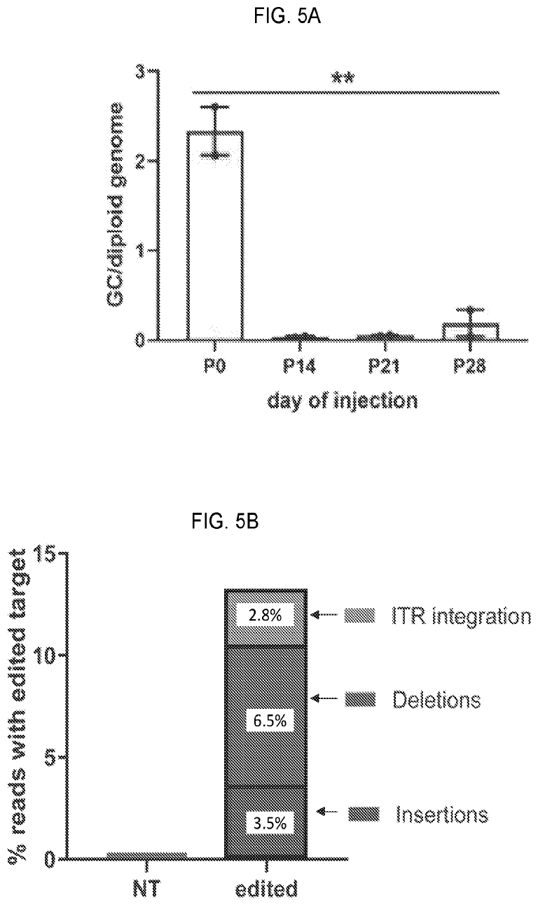

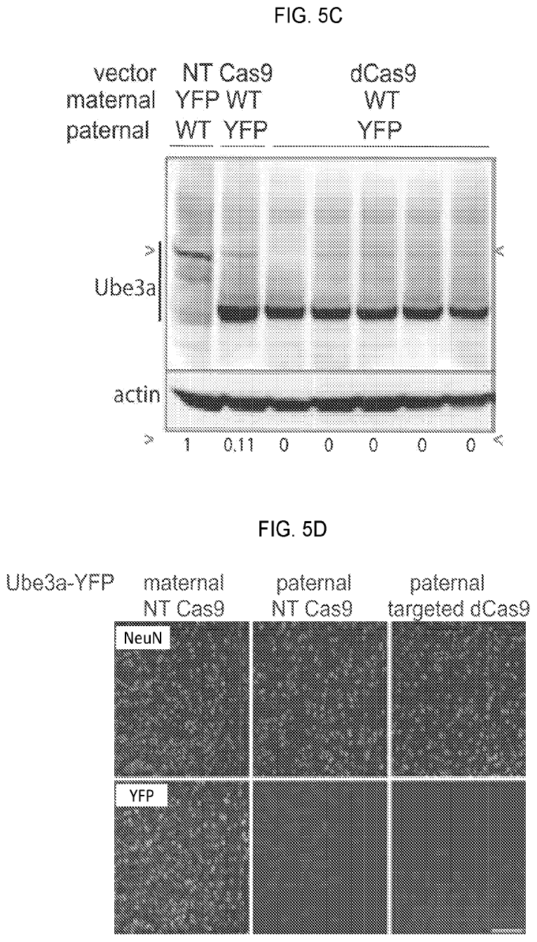

To introduce indels into Ube3α-ATS, we initially screened 12 single-guide RNAs (sgRNAs) that target different sites within a 12 kb segment of the Ube3α-ATS coding sequence (FIG. 2A, Table 2). We aimed to select targets outside of known expressed gene loci. Cas9-induced indels at the respective target sites were assessed by Amplicon-seq (FIG. 2B). We then constructed an adeno-associated virus (AAV) plasmid harboring sgRNA #7 (FIG. 2B) and the S. aureus Cas9 coding sequence. We used the human synapsin promoter (13, 14) to selectively drive Cas9 expression in neurons in the mouse brain. Delivery of the AAV gene editing vector—which we refer to as ATS-GE—to the neonatal mouse brain via intracerebroventricular (ICV) injection resulted in the formation of genomic indels in 14.7% (8.6-21.7%) of all brain cells (FIG. 2C). When we expressed either a non-targeting (NT) sgRNA or a nuclease-deficient Cas9 (dCas9), indel formation remained at background frequencies (0.17% or 0.04%, respectively; FIG. 2C). Indel formation was highest after neonatal ICV vector delivery. The PHP.B capsid facilitates very efficient transduction of the mouse brain via intravenous (IV) delivery at any age (15). However, we observed much lower indel frequencies when ATS-GE was delivered at 14-28 days of age (0.6-2.9%, FIG. 2C), suggesting that ICV injection into the newborn mouse brain is best suited for efficient gene editing for this project. In support of this notion, the amount of vector genome present in brain tissue after ICV injection into the newborn brain was much higher [average of 2.3 genome copies (GC) per diploid genome] compared to transduction after IV injection at post-natal days 14, 21 or 28 (0.04 to 0.19 GC per diploid genome, FIG. 5A). Additional analysis of the target site using anchored multiplexed PCR sequencing (AMP-Seq) showed that most editing events following neonatal ICV delivery were indeed short indels of less than 15 bp; integration of vector sequence portions at the editing site only was observed at a frequency of 2.8% (FIG. 5B). Computational analysis predicted no additional direct matches for our chosen sgRNA throughout the mouse genome, with the nearest similarities including at least four mismatches. Accordingly, inverted terminal repeat sequencing (ITR-Seq) revealed only a small genome-wide off-target rate (1.3%) for targeted editing with ATS-GE (Table 3). We found that indels were present at similar frequencies when we performed molecular analyses on cortical cells 3, 8 or 14 weeks after neonatal ATS-GE vector delivery (FIG. 2D). This result suggests that neurons with an edited Ube3α-ATS sequence persist in the adult mouse brain.

TABLE 2
Sequences and indel frequencies for in
vitro screened sgRNA. Indel% were
determined by Amplicon-seq at the 12
target sites using either a non-
targeting (NT) sgRNA or the respective
sgRNA. sgRNA #7 was chosen for in vivo
follow-up.

| sgRNA | | NT | targeted |
|---|---|---|---|
| # | target sequence | indel % | indel% |
| 1 | CATGAAGGTAACCACTACTG | 0.04 | 22.11 |
| | (SEQ ID NO: 42) | | |
| 2 | CCTGGCCTTCCCTGTATCAA | 0.02 | 8.00 |
| | (SEQ ID NO: 43) | | |
| 3 | CAAATTTGGGCCTTGGTGTC | 0.01 | 5.13 |
| | (SEQ ID NO: 44) | | |
| 4 | GAATGTTGGGATCTCAAAC | 1.58 | 0.01 |
| | (SEQ ID NO: 45) | | |
| 5 | GTGCTGTTTTGCAAACCTTTA | 0.01 | 0.01 |
| | (SEQ ID NO: 46) | | |
| 6 | TCCTTGGGCCTCCTATGTCA | 0.01 | 0.24 |
| | (SEQ ID NO: 47) | | |
| 7 | TTGCCCAACCTCTCAAACGT | 0.01 | 32.97 |
| | (SEQ ID NO: 48) | | |
| 8 | CAGGCCTTGGTGTTATGAAT | 0.01 | 19.60 |
| | (SEQ ID NO: 49) | | |
| 9 | TTGGTACTTACAAGGCCATG | 0.02 | 16.84 |
| | (SEQ ID NO: 50) | | |
| 10 | TCACACAAGAGCCACCCCA | 0.73 | 28.88 |
| | (SEQ ID NO: 51) | | |
| 11 | CTGAGGCCCATCCATGGTCA | 0.03 | 36.023 |
| | (SEQ ID NO: 52) | | |
| 12 | GCCTCATGTGACCCTGTGACC | 0.02 | 5.16 |
| | (SEQ ID NO: 53) | | |

TABLE 3
In vivo off-target analysis for gene editing with selected sgRNA.
Ube3a^{m+/pYFP} (paternal Ube3a-YFP) mice were injected with an
AAV vector encoding CRISPR/Cas9 at birth (day 0), and the cerebral
cortices were harvested 21 days later. ITR-seq was carried out
for detecting off-target gene editing (three mice per group)

| | | On- target | | Off- target |
|---|---|---|---|---|
| Group | On-target | location(s) | Off-target | location(s) |
| Non-targeted | 0% | — | 100% | chr2: 98666606 |
| (NT) Cas9 | 0 reads | | 134 reads | chr6: 5403525 |
| vector | | | | chr6: 48598280 |
| Targeted | 98.7% | chr7: 59346117 | 1.3% | chr2: 98667110 |
| Cas9 vector | 5001 reads | | 66 reads | chr8: 116314485 |
| | | | | chr9: 3024429 |
| | | | | chr9: 112134613 |

Ube3α-ATS interferes with the extension of the Ube3a transcript on the paternal allele, blocking Ube3a expression from the paternal allele. A promising therapeutic approach for AS relies on abrogating the extension of Ube3α-ATS across the Ube3a gene locus on the paternal allele to allow for full-length Ube3a transcript formation and thus protein expression. Thus, we next evaluated whether the observed indel formation in our studies could suppress extension of Ube3α-ATS across the Ube3a paternal allele. To unambiguously detect Ube3a expression from the paternal allele, we crossed wild-type females with male mice harboring an sn fusion gene (16). Newborn pups ICV injected with the ATS-GE vector showed expression of the Ube3α-YFP fusion protein 21 days later (FIG. 2E, FIG. 2F). Immunofluorescence staining revealed expression of Ube3α-YFP throughout the cortex after gene editing in 48.1% of neurons (FIG. 2G, and quantification of 13,000 NeuN⁺ cells, SEM=8.6%). When we replaced the targeting sgRNA in the ATS-GE vector with a non-targeting sgRNA, or replaced Cas9 with dCas9, we observed no Ube3α-YFP expression (FIG. 5D and FIG. 5D). Molecular analysis revealed that Ube3α-ATS expression from the paternal allele was reduced within the Ube3a gene locus (FIG. 2H). By contrast, expression of genes located between the gene editing location and the imprinting center (IC) was unaffected (Snrpn, Snord115, or Snord116, FIG. 2I). Thus, selective and efficient gene editing within the Ube3α-ATS leads to Ube3a protein expression from the previously silenced paternal Ube3a allele.

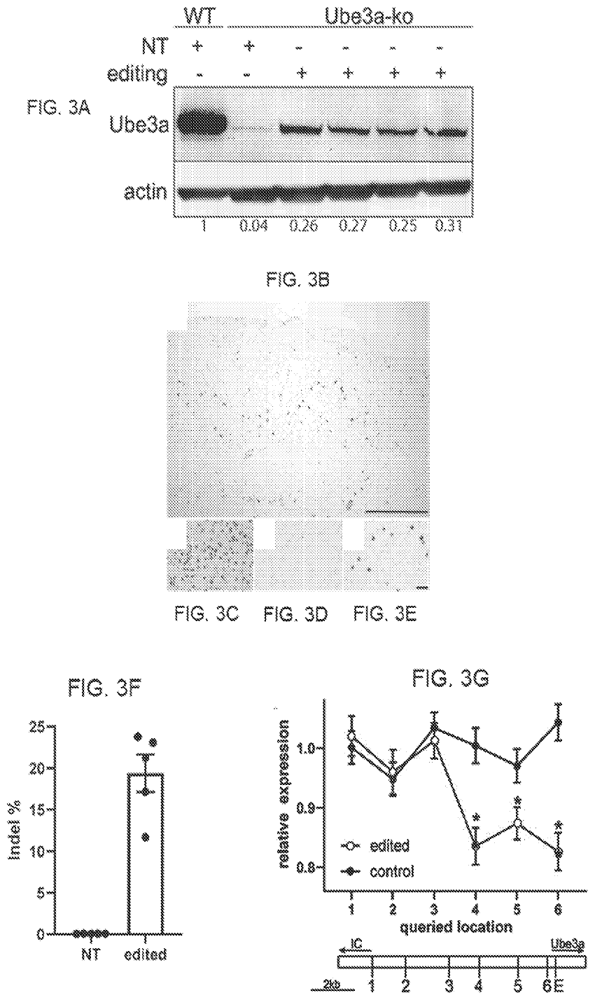

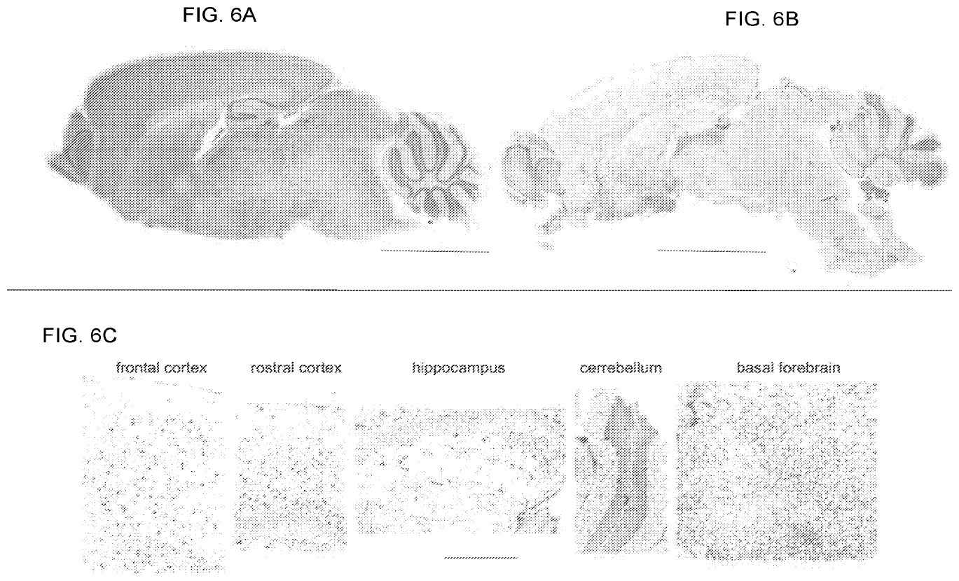

Next, we investigated whether Ube3α-ATS gene editing could drive expression of Ube3a from the paternal allele to restore Ube3a expression in neurons of maternal Ube3α-knockout (KO) mice and improve the mouse AS phenotype. We administered the ATS-GE vector to neonatal maternal Ube3α-KO pups and wild-type littermates via ICV injection. We then observed the expression of Ube3a protein throughout the brain after four months of incubation (FIG. 3A). We detected expression of Ube3a in neurons throughout the brain (FIG. 3B and FIG. 6A). We observed Ube3α-positive neurons throughout the brain (FIG. 6B, FIG. 6C), and it seemed that expression was more abundant in the basal forebrain regions. A larger, more comprehensive study is needed to investigate whether expression efficiency varies among brain regions and functional substructures, but this was beyond the scope of the current investigation.

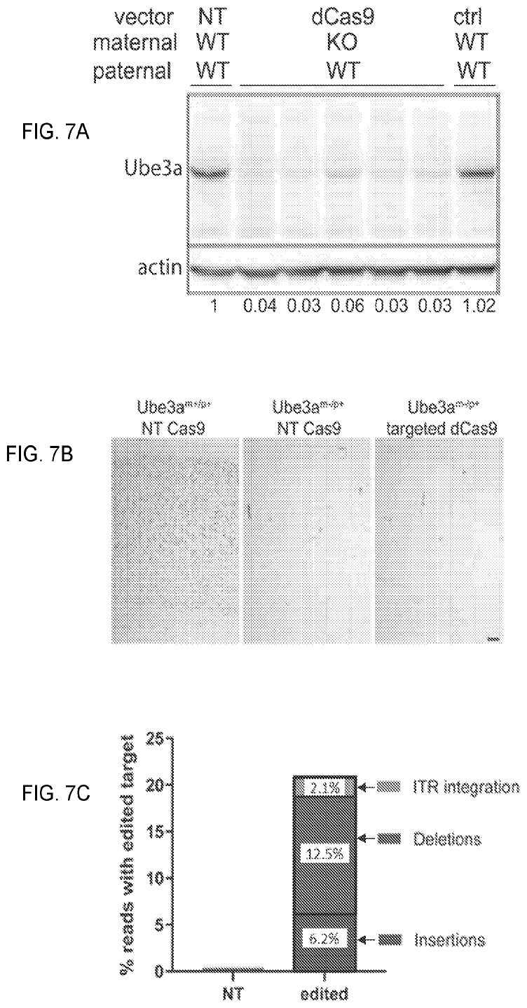

Mice that received the control AAV vector (harboring non-targeted Cas9 or targeted dCas9 sequences) did not show Ube3a expression from the paternal allele (FIG. 7A and FIG. 7B). Molecular analysis by Amplicon-seq revealed that indel frequencies occurred at an average of 19.4% (FIG. 3F), which is comparable to observations from the previous short-term study (FIG. 2C). Vector integrations at the gene-editing site were low (AMP-seq, 2.1%, FIG. 7C) as previously observed (FIG. 5A). Off-target analysis by ITR-seq did not show an increased rate of off-target effects (Table 1) compared to the previous short-term study (Table 3). The only two identified off-target sites were located in an intron or an unannotated genomic region. ATS-Ube3a transcript levels were significantly reduced in Ube3α-KO mouse brains after gene editing (FIG. 3G). The transcript levels were found to have normalized about 4 kb from the gene-editing site towards the imprinting center.

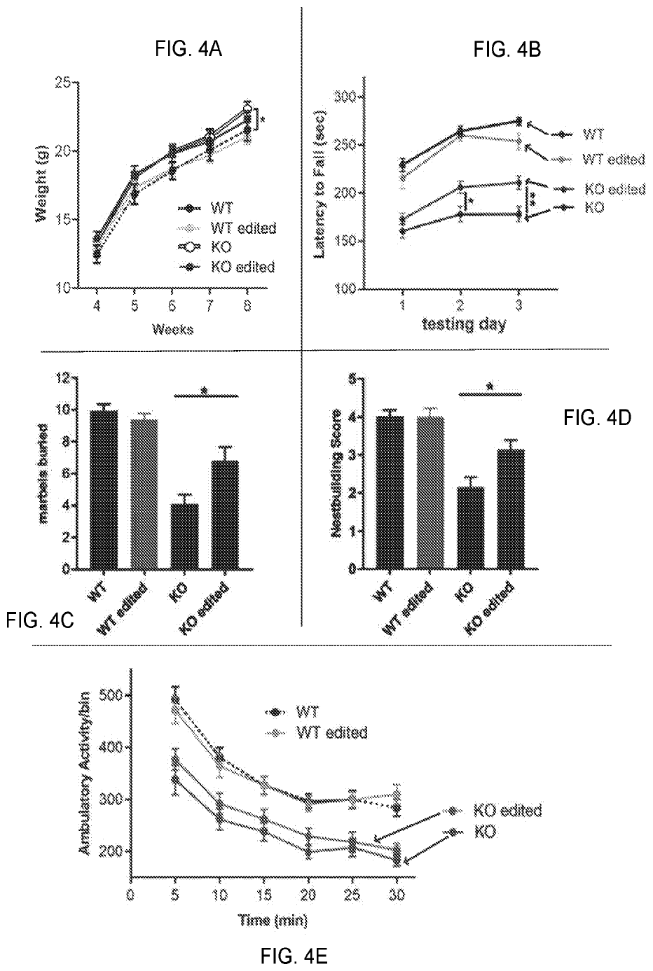

The behavioral phenotype of maternal Ube3α-KO mice has been well characterized and can be improved by genetically restoring maternal Ube3a expression. Treatment with ASOs transiently suppresses the extension of Ube3α-ATS across the Ube3a locus, leading to paternal Ube3a expression in neurons throughout the brain and the subsequent improvement of the behavioral phenotype. This approach restores Ube3a expression in a much larger number of neurons throughout the mouse brain, so we were wondering whether gene editing of Ube3α-ATS in a limited number of neurons could improve the maternal Ube3α-KO phenotype. We ICV-injected neonatal mice with ATS-GE vector and monitored them until four months of age. Ube3α-ATS gene editing was tolerated well with no treatment-related mortalities. As expected, weight gain was significantly higher in AS mice and showed a trend to reduction after ATS-GE treatment during the observation period (FIG. 4A). At two months of age, the mice were subjected to a sequence of behavioral tests that have been widely used with this mouse model (17). Maternal Ube3α-KO mice showed the expected significant deficits in motor function in comparison to their wild-type littermates when tested with a rotarod (FIG. 4B). Gene-edited maternal Ube3α-KO mice showed a significant improvement of motor function on testing days two and three (FIG. 4B). Similarly, marble burying and nest-building behaviors were impaired in maternal Ube3α-KO mice, but were significantly improved after Ube3α-ATS gene editing (FIG. 4C, FIG. 4D). Ambulatory activity in the open-field test showed a modest yet consistent trend toward improvement of hypoactivity in maternal Ube3α-KO mice after Ube3α-ATS gene editing (FIG. 4E). In summary, restoring Ube3a expression by suppressing Ube3α-ATS in a subset of neurons significantly improved multiple behavioral aspects of the maternal Ube3α-KO phenotype. It remains to be investigated whether Ube3α-ATS gene editing improves other maternal Ube3α-KO mouse phenotypes, such as electroencephalogram alterations.

This study demonstrated two important findings: (i) efficient gene editing can be achieved in the mouse brain by neonatal intraventricular AAV delivery; and (ii) expression of Ube3a in a subset of neurons (a maximum of 20% based on sequencing data) provides a therapeutic benefit in an AS mouse model. Nuclease activity of Cas9 was necessary to achieve Ube3a protein expression since expression of inactive Cas9 (dCas9) did not cause Ube3a protein expression.

The CNS has been recognized as promising target for therapeutic genome editing, particularly since disruption of a pathological allele holds promise for curative treatment of genetic disorders (18-20). Recent studies for therapeutic CNS gene editing have achieved promising results via focal delivery of CRISPR/Cas9 complex, e.g., into the striatum of a Huntington's disease mouse model (21), into the spinal cord of an amyotrophic lateral sclerosis mouse model (22), or into the hippocampus of a mouse model of familial Alzheimer's disease (23). However, it remains to be shown whether CRISPR/Cas9 can successfully edit a sufficient number of neurons to achieve a therapeutic benefit in human patients if editing throughout different brain regions needs to be achieved. Our study demonstrates that this is possible in the mouse brain and can result in the significant improvement of a disease phenotype. For AS, as for many other genetic CNS disorders, we know little about the required efficiency of disease gene re-expression to cause a therapeutic benefit. Our study suggests that re-expression of Ube3a is not required in all neurons. Previous studies that used genetic reinstatement of Ube3a or ASO-mediated Ube3a expression showed a larger effect size of behavior improvement due to the much large number of neurons expressing Ube3a (8, 11, 12). Our studies are encouraging for further translational and clinical AS research since inefficient expression of UBE3A may already be enough to translate into a therapeutic effect in patients. A recent study with CRISPR/Cas9-mediated replacement of a 245 kb section within the Ube3α-ATS with an AAV vector insertion also reported limited neurobehavioral improvement in AS model mice (24).

Our study does not directly address a potential mechanism for how indel formation results in a shortened Ube3α-ATS that selectively enables lasting paternal Ube3a expression. We can only speculate that the secondary or tertiary genomic structure of the Ube3a locus may play a role in the antagonistic gene expression regulation of Ube3a and Ube3α-ATS. Alternatively, or additionally, we speculate that the extremely long paternal Ube3α-ATS transcript may be prone to early termination in the presence of paternal sense Ube3a transcript. Gene editing may cause an initial transcriptional pausing of the Ube3α-ATS transcript, which allows formation of the Ube3a sense transcript, with this situation persisting even after double-strand repair has been completed. Another possible mechanism of action includes the ability of S. aureus Cas9 to cleave RNA transcripts (25), which likely would require constitutive expression of Cas9 to maintain Ube3a expression; we did not, however, observe sustained Cas9 expression in all Ube3α-expressing AS mouse brains (data not shown). Lastly, integration of AAV vector sequence could lead to premature termination of Ube3α-ATS, as observed in a recent study (24). Given that total detected integrations remained at 2-3% over 4 months, this mechanism could contribute to, but unlikely be solely responsible for all, detected Ube3a expression.

Although the genomic organization and regulation of Ube3α-ATS and the imprinting control center are highly conserved between mouse and human (11), the DNA sequence is very different for these two species. A redesign of the mouse sgRNA targeting a similar genomic location in humans would be a necessary prerequisite for translational studies. One would expect that interference of UBE3A ATS by gene editing could similarly restore UBE3A expression in the neurons of AS patients.

### REFERENCES

1. Buiting K, Williams C, and Horsthemke B. Angelman syndrome—insights into a rare neurogenetic disorder. Nature reviews Neurology. 2016.
2. Dagli A, Buiting K, and Williams C A. Molecular and Clinical Aspects of Angelman Syndrome. Mol Syndromol. 2012; 2(3-5):100-12.
3. Albrecht U, Sutcliffe J S, Cattanach B M, Beechey C V, Armstrong D, Eichele G, et al. Imprinted expression of the murine Angelman syndrome gene, Ube3a, in hippocampal and Purkinje neurons. Nat Genet. 1997; 17(1):75-8.
4. Kishino T, Lalande M, and Wagstaff J. UBE3A/E6-AP mutations cause Angelman syndrome. Nat Genet. 1997; 15(1):70-3.
5. Matsuura T, Sutcliffe J S, Fang P, Galjaard R J, Jiang Y H, Benton C S, et al. De novo truncating mutations in E6-AP ubiquitin-protein ligase gene (UBE3A) in Angelman syndrome. Nat Genet. 1997; 15(1):74-7.
6. Keute M, Miller M T, Krishnan M L, Sadhwani A, Chamberlain S, Thibert R L, et al. Angelman syndrome genotypes manifest varying degrees of clinical severity and developmental impairment. Mol Psychiatry. 2020.
7. Chamberlain S J, and Brannan C I. The Prader-Willi syndrome imprinting center activates the paternally expressed murine Ube3a antisense transcript but represses paternal Ube3a. Genomics. 2001; 73(3):316-22.
8. Meng L, Person R E, Huang W, Zhu P J, Costa-Mattioli M, and Beaudet A L. Truncation of Ube3α-ATS unsilences paternal Ube3a and ameliorates behavioral defects in the Angelman syndrome mouse model. PLoS Genet. 2013; 9(12):e1004039.
9. Rougeulle C, Cardoso C, Fontes M, Colleaux L, and Lalande M. An imprinted antisense RNA overlaps UBE3A and a second maternally expressed transcript. Nat Genet. 1998; 19(1):15-6.
10. Huang H S, Allen J A, Mabb A M, King I F, Miriyala J, Taylor-Blake B, et al. Topoisomerase inhibitors unsilence the dormant allele of Ube3a in neurons. Nature. 2012; 481(7380):185-9.
11. Meng L, Ward A J, Chun S, Bennett C F, Beaudet A L, and Rigo F. Towards a therapy for Angelman syndrome by targeting a long non-coding RNA. Nature. 2015; 518(7539):409-12.
12. Silva-Santos S, van Woerden G M, Bruinsma C F, Mientj es E, Jolfaei M A, Distel B, et al. Ube3a reinstatement identifies distinct developmental windows in a murine Angelman syndrome model. J Clin Invest. 2015; 125(5):2069-76.
13. Kugler S, Kilic E, and Bähr M. Human synapsin 1 gene promoter confers highly neuron-specific long-term transgene expression from an adenoviral vector in the adult rat brain depending on the transduced area. Gene Therapy. 2003; 10(4):337-47.
14. Thiel G, Greengard P, and Sudhof T C. Characterization of tissue-specific transcription by the human synapsin I gene promoter. Proc Natl Acad Sci USA. 1991; 88(8):3431-5.
15. Deverman B E, Pravdo P L, Simpson B P, Kumar S R, Chan K Y, Banerjee A, et al. Cre-dependent selection yields AAV variants for widespread gene transfer to the adult brain. Nat Biotechnol. 2016; 34(2):204-9.
16. Dindot S V, Antalffy B A, Bhattacharjee M B, and Beaudet A L. The Angelman syndrome ubiquitin ligase localizes to the synapse and nucleus, and maternal deficiency results in abnormal dendritic spine morphology. Hum Mol Genet. 2008; 17(1):111-8.
17. Sonzogni M, Wallaard I, Santos S S, Kingma J, du Mee D, van Woerden G M, et al. A behavioral test battery for mouse models of Angelman syndrome: a powerful tool for testing drugs and novel Ube3a mutants. Molecular autism. 2018; 9:47.
18. van Haasteren J, Li J, Scheideler O J, Murthy N, and Schaffer D V. The delivery challenge: fulfilling the promise of therapeutic genome editing. Nat Biotechnol. 2020; 38(7):845-55.
19. Cox D B, Platt R J, and Zhang F. Therapeutic genome editing: prospects and challenges. Nat Med. 2015; 21(2):121-31.
20. Nishiyama J. Genome editing in the mammalian brain using the CRISPR-Cas system. Neurosci Res. 2019; 141:4-12.
21. Ekman F K, Ojala D S, Adil M M, Lopez P A, Schaffer D V, and Gaj T. CRISPR-Cas9-Mediated Genome Editing Increases Lifespan and Improves Motor Deficits in a Huntington's Disease Mouse Model. Mol Ther Nucleic Acids. 2019; 17:829-39.
22. Gaj T, Ojala D S, Ekman F K, Byrne L C, Limsirichai P, and Schaffer D V. In vivo genome editing improves motor function and extends survival in a mouse model of ALS. Sci Adv. 2017; 3(12):eaar3952.
23. Gyorgy B, Loov C, Zaborowski M P, Takeda S, Kleinstiver B P, Commins C, et al. CRISPR/Cas9 Mediated Disruption of the Swedish APP Allele as a Therapeutic Approach for Early-Onset Alzheimer's Disease. Mol Ther Nucleic Acids. 2018; 11:429-40.
24. Wolter J M, Mao H, Fragola G, Simon J M, Krantz J L, Bazick H O, et al. Cas9 gene therapy for Angelman syndrome traps Ube3α-ATS long non-coding RNA. Nature. 2020.
25. Strutt S C, Torrez R M, Kaya E, Negrete O A, and Doudna J A. RNA-dependent RNA targeting by CRISPR-Cas9. eLife. 2018; 7.

### Sequence Listing Free Text

The following information is provided for sequences containing free text under numeric identifier <223>.

| SEQ ID NO: | Free text under <223> |
|---|---|
| 1-32 | <223> sgRNA sequence |
| 33-34 | <223> miR target sequence |
| 36-41 | <223> primer sequence |
| 42-53 | <223> target sequence |
| 56 | <223> non-targeted sequence |

All documents cited in this specification are incorporated herein by reference. The sequence listing filed herewith named “20-9231PCT_ST25” and the sequences and text therein are incorporated by reference. U.S. Provisional Application No. 63/016,712, filed Apr. 28, 2020, and U.S. Provisional Application No. 63/118,299, filed Nov. 25, 2020, are incorporated herein by reference. While the invention has been described with reference to particular embodiments, it will be appreciated that modifications can be made without departing from the spirit of the invention. Such modifications are intended to fall within the scope of the appended claims.

1. An expression cassette comprising a nucleic acid sequence encoding one or more elements of a gene editing system that targets and introduces a mutation in UBE3A-ATS on a paternal allele in a neuron of a patient having Angelman syndrome and regulatory elements that direct expression thereof in a target cell, thereby unsilencing the paternal UBE3A allele and permitting expression of the UBE3A gene product.

2. The expression cassette according to claim 1, wherein the gene editing system is CRISPR/Cas, a meganuclease, a zinc-finger nuclease, or a TALEN.

3. The expression cassette according to claim 1, wherein the nucleic acid encodes a gene editing nuclease and/or a targeting sequence specific for UBE3A-ATS.

4. The expression cassette according to claim 1, wherein the gene-editing system comprises a CRISPR-associated nuclease and an sgRNA having a sequence that specifically binds a UBE3A-ATS target sequence.

5. The expression cassette according to claim 4, wherein the CRISPR endonuclease is Cas9, optionally SaCas9.

6. The expression cassette according to claim 1, wherein the expression cassette comprises a sequence encoding any of SEQ ID NOs: 1-32.

7. The expression cassette according to claim 1, wherein
(a) the UBE3A-ATS target sequence is downstream of the UBE3A 3′UTR; and/or
(b) the target sequence is located at chr15: 25,278,409-25,333,728 (hg38 genome assembly) and/or in a sequence of UBE3A-ATS complementary to the region between the UBE3A 3′UTR and SNORD109B ORF on chromosome 15.

8. (canceled)

9. The expression cassette according to claim 1, wherein the target cell is a cell of the CNS.

10. The expression cassette according to claim 1, wherein the regulatory elements comprise
(a) a neuron-specific promoter, optionally wherein the promoter is a synapsin promoter; and/or
(b) an enhancer.

11.-12. (canceled)

13. A plasmid comprising the expression cassette according to claim 1.

14. (canceled)

15. A recombinant adeno-associated virus (rAAV) useful as a CNS-directed therapeutic for treatment of Angelman syndrome (AS), the rAAV comprising an AAV capsid, and a vector genome packaged therein, said vector genome comprising:
(a) an AAV 5′ inverted terminal repeat (ITR);
(b) a nucleic acid sequence encoding one or more elements of a gene editing system that targets UBE3A-ATS;
(c) regulatory elements that direct expression of the one or more elements of the gene editing system; and
(d) an AAV 3′ ITR.

16. The rAAV according to claim 15, wherein the gene targeting system comprises a CRISPR endonuclease and a sgRNA that specifically binds a UBE3A-ATS target sequence.

17. The rAAV according to claim 15 or 16, wherein the CRISPR endonuclease is Cas9.

18. The rAAV according to claim 15, wherein the regulatory elements comprise
(a) a neuron-specific promoter, optionally wherein the promoter is a synapsin promoter; and/or
(b) an enhancer.

19. (canceled)

20. The rAAV according to claim 15, wherein the capsid is an AAV9 capsid, or variant thereof, or an AAVhu68 capsid, or variant thereof.

21. The rAAV according to claim 15, wherein the AAV capsid is an AAVhu68 capsid generated from expression of the nucleic acid sequence of SEQ ID NO: 54.

22. A pharmaceutical composition comprising at least the rAAV according to claim 15 and a physiologically compatible carrier, buffer, adjuvant, and/or diluent.

23. (canceled)

24. A method for treating one or more symptoms of Angelman syndrome (AS) in a patient having deficient UBE3A expression in neurons, said method comprising delivering a nucleic acid sequence that encodes one or more elements of a gene editing system that targets a sequence in UBE3A-ATS downstream of the UBE3A 3′UTR to modify the UBE3A-ATS coding sequence, thereby unsilencing UBE3A expression on a paternal allele of a patient having a deficiency in UBE3A expression from a maternal allele and providing for expression of the UBE3A gene product from the paternal allele.

25. The method according to claim 24, wherein
(a) the modification is an indel, deletion, insertion, inversion, or other disruption in the UBE3A-ATS coding sequence;
(b) the modification is introduced in the human UBE3A-ATS in the region spanning the UBE3A 3′UTR and SNORD109B; and/or
(c) the target sequence is located at chr15: 25,278,409-25,333,728 (hg38 genome assembly) and/or in a sequence of UBE3A-ATS complementary to the region between the UBE3A 3′UTR and SNORD109B ORF on chromosome 15.

26.-27. (canceled)

28. The method according to claim 24, wherein the symptoms are selected from one or more of: delayed development, intellectual disability, severe speech impairment, ataxia and/or epilepsy.

29. (canceled)
